SEQUENCE LISTING (1 sequence)

SEQ ID NO: 1 - DNA, 1317 bp, Gallus gallus
agatctgccaccatgaggcttcctggttggttgtggctgagttctgccgtcctcgctgcctgccgagcggtggaggagcacaacctgactgaggggctggaggatgccagcgcccaggctgcctgccccgcgaggctggagggcagcgggaggtgcgaggggagccagtgccccttccagctcaccctgcccacgctgaccatccagctcccgcggcagcttggcagcatggaggaggtgctcaaagaagtgcggaccctcaaggaagcagtggacgctctgaagaaatcctgccaggactgtaagttgcaggctgacgaccatcgagatcccggcgggaatggagggaatggagcagagacagccgaggacagtagagtccaggaactggagagtcaggtgaacaagctgtcctcagagctgaagaatgcaaaggaccagatccaggggctgcaggggcgcctggagacgctccatctggtaaatatgaacaacattgagaactacgtggacaacaaagtggcaaatctaaccgttgtggtcaacagtttggatggcaagtgttccaagtgtcccagccaagaacacatgcagtcacagccggttcaacatctaatatacaaagattgttccgaccactacgtgctaggaaggagaagcagtggggcctacagagttacccctgatcacagaaacagcagctttgaggtctactgtgacatggagaccatgggtggaggctggacggtgctgcaggctcgccttgatggcagcaccaacttcaccagagagtggaaagactacaaagccggctttggaaaccttgaacgagaattttggttgggcaacgataaaattcatcttctgaccaagagtaaggaaatgattttgagaatagaccttgaagactttaatggtctcacactttatgccttgtatgatcagttttatgtggctaatgaatttctcaaataccgattacacatcggtaactacaatggcacgggaggggatgccttgcgtttcagtcgacactacaaccatgacctgaggtttttcacaaccccagacagagacaacgatcggtacccctctgggaactgtgggctctattacagctcaggctggtggtttgattcatgtctctctgccaacttaaatggcaaatattaccaccagaaatacaaaggtgtccgtaatgggattttctggggcacctggcctggtataaaccaggcacagccaggtggctacaagtcctccttcaaacaggccaagatgatgattaggcccaagaatttcaagccataactcgag

(19) United States
(12) Patent Application Publication
(10) Pub. No.: US 2017/0258834 A1
(43) Pub. Date: Sep. 14, 2017

(54) FGL2 Expressing Regulatory T Cells

(71) Applicant: Veritas Therapeutics Inc., Thornhill (CA)

(72) Inventors: Gary A. Levy, Thornhill (CA); David Grant, Toronto (CA); Andrzej Chruscinski, Toronto (CA)

(73) Assignee: Veritas Therapeutics Inc., Thornhill (CA)

(21) Appl. No.: 15/456,656

(22) Filed: Mar. 13, 2017

Related U.S. Application Data
(60) Provisional application No. 62/307,784, filed on Mar. 14, 2016.

(51) Int. Cl. A61K 35/17; A61K 45/06; C07K 14/47; A01K 67/027

(52) U.S. Cl. CPC A61K 35/17; A01K 67/0278; A61K 45/06; C07K 14/47; A01K 2267/01; A01K 2227/105; A01K 2217/072; A61K 2035/124

(57) ABSTRACT

This application relates to methods and compositions for inducing immune tolerance. Specifically, methods and uses of regulatory T cells (Treg) and associated compositions for the induction of immune tolerance are described. The methods and uses may be used to prevent transplant rejection, and in the treatment of diseases or conditions such as graft versus host disease, autoimmune disease and allergies. Also provided are transgenic mice that ubiquitously express FGL2 protein from which the Treg may be isolated.

This application claims the benefit under 35 USC §119(e) from U.S. Provisional patent application Ser. No. 62/307,784, filed Mar. 14, 2016 which is incorporated herein by reference.

### INCORPORATION OF SEQUENCE LISTING

A computer readable form of the Sequence Listing “25306-P50264US01_SequenceListing.txt” (4,096 bytes), submitted via EFS-WEB and created on Mar. 10, 2017, is herein incorporated by reference.

### FIELD

The present disclosure relates to methods and compositions for inducing immune tolerance. Specifically, methods and uses of regulatory T cells (Treg) and associated compositions for the induction of immune tolerance are described. The methods and uses may be used to prevent transplant rejection and in the treatment of diseases or conditions where it is desirable to suppress an immune response including graft versus host disease, autoimmune disease and allergies. Also provided are transgenic mice that ubiquitously express FGL2 protein from which the Treg may be isolated.

### BACKGROUND

Solid organ transplantation is recognized as one of the major medical achievements of the last half-century (1, 2). However, the success of transplantation is limited by the need for long-term immunosuppression, which is associated with significant patient mortality and complications including viral and bacterial opportunistic infections, diabetes mellitus, cardiovascular disease and cancer (3-6). The ability to induce transplant tolerance would allow for the withdrawal of immunosuppression, thereby improving long-term patient survival (7, 8).

Regulatory T cells (Treg) have been shown to play a critical role in tolerance induction as shown by the finding that depletion of Treg prevents the development of tolerance in experimental animal models (9-12). Treg employ multiple mechanisms to suppress immune responses including expression of suppressive cytokines (e.g., IL-10, IL-35 and TGF-13), altering effector cell migration, binding of IL-2 by the high affinity IL-2 receptor (13), and decreasing cell metabolism in T cells (13-17). Over the last decade, several Treg effector molecules involved in suppression have been identified including CD39, lymphocyte activation gene (LAG)-3, cytotoxic T lymphocyte antigen (CTLA)-4 and, most recently, fibrinogen-like protein 2 (FGL2) (18).

FGL2 is an immunoregulatory protein that has roles in both innate and adaptive immune responses. As part of the innate immune system, FGL2 exists as a membrane-bound protein, which has direct prothrombinase activity (21). The immune coagulation activity of FGL2 promotes fibrin deposition and has been implicated in the pathogenesis of acute and chronic viral hepatitis, fetal loss syndrome, and xenograft rejection (20, 45, 46). The secreted form of FGL2, on the other hand, has been shown to have immunoregulatory activity (19).

FGL2 is a multifunctional protein that has been shown to have prothrombinase activity in addition to immunoregulatory activity (19, 20). FGL2 is a member of the fibrinogen related family of proteins whose members all contain a fibrinogen-related domain (FReD) (21). Several proteins in this family (e.g., tenascin, angiopoietin, fibrinogen and ficolin) have immunoregulatory or immunosuppressive activity in addition to their roles in coagulation and angiogenesis (47, 48). The immunoregulatory activity has been mapped to the FReD region and, in the case of FGL2, is exerted through binding to low affinity Fcγ receptors, FcγRIIB/RIII expressed on macrophages, B cells and dendritic cells (DC) (19). It has been demonstrated that recombinant FGL2 (rFGL2) prevents the maturation of DC, induces B cell apoptosis and suppresses T cell proliferation in vitro (22, 23). Multiple investigators have also shown FGL2 expression in Treg (13, 18, 19, 22, 24, 25) and other regulatory cells including CD8αα, double-negative and CD8⁺CD45RC^{low}T cells (11, 18, 26). Recently, FGL2 was shown to be an important effector of a highly suppressive Treg subset that expresses T cell immunoglobulin and ITIM domain (TIGIT) (27).

T and B cells from fgl2 deficient mice (fgl2^{-/-}) have enhanced activation, and fgl2^{-/-} Treg have reduced suppressive activity (23). It has also been recently found that fgl2 expression was increased in tolerant heart allografts induced by rapamycin and an antibody to FGL2 prevented the development of tolerance (28). However, there remains a need to further elucidate the role of Treg in inducing immune tolerance which may aid in the development of novel therapies.

### SUMMARY

The ability to induce immune tolerance could assist in the treatment of disorders or conditions where it is desirable to suppress an immune response including allo and autoimmune diseases such as transplantation, inflammatory bowel disease, diabetes mellitus and rheumatoid arthritis. The data disclosed herein demonstrates that recombinant FGL2 (rFGL2) treatment or constitutive FGL2 overexpression could promote transplant tolerance in mice, and further that regulatory T cells (Treg) modified to overexpress FGL2 exhibit increased immunosuppressive activity compared to unmodified Treg.

Accordingly, the present disclosure provides a regulatory T cell (Treg) comprising a recombinant fgl2 transgene. In one embodiment, the transgene is operably connected to a promoter that drives high levels of gene expression. In another embodiment, the fgl2 transgene encodes a FGL2 protein that lacks prothrombinase activity. In a further embodiment, the fgl2 transgene comprises a nucleic acid sequence with at least 80%, 85%, 90%, 95%, 96%, 97%, 98%, 99% or 100% sequence identity to SEQ ID NO:1 which has been operably connected to a CMV early enhancer/chicken beta actin (CAG) promoter. In an additional embodiment, the Treg is isolated from a transgenic mouse disclosed herein.

Also provided herein is a composition comprising the Treg and a pharmaceutically acceptable carrier. In another embodiment, the composition further comprises one or more immunosuppressive agents. The composition may be used, for example, to induce immune tolerance or to suppress an immune response.

In one embodiment, the present disclosure provides a method of inducing tolerance in a subject in need thereof comprising administering to the subject a therapeutically effective amount of a Treg or composition disclosed herein. The present disclosure also provides a use of a therapeutically effective amount of a Treg or composition disclosed herein for inducing tolerance in a subject in need thereof. Further provided is a use of an effective amount of a Treg or composition disclosed herein in the preparation of a medicament for inducing tolerance in a subject in need thereof. Even further provided is an effective amount of a Treg or composition disclosed herein for use in inducing tolerance in a subject in need thereof.

The subject may be a mammal, optionally a human.

The methods of the disclosure may be used to treat any disease or condition where it is desirable to induce tolerance or to suppress an immune response. This may include preventing the rejection of transplanted organs or tissues and for treating graft versus host disease, autoimmune disease and allergies.

Accordingly, in one embodiment, tolerance is induced to a transplanted organ or tissue. In a further embodiment, tolerance is induced in order to treat graft versus host disease. In an additional embodiment, tolerance is induced in order to treat an autoimmune disease or disorder. In another embodiment, tolerance is induced in order to treat an allergic reaction.

The present disclosure also provides a transgenic mouse referred to herein as a “fgl2^{Tg} mouse” that has been engineered to overexpress a fgl2 transgene that has been modified such that the prothrombinase active site of the FGL2 protein is mutated from Ser to Ala in order to eliminate prothrombinase activity. As shown herein, Fgl2^{Tg} mice ubiquitously express FGL2 such that plasma levels of FGL2 were determined to be ˜7-fold higher in fgl2^{Tg} mice than in fgl2^{+/+} mice. It has also been surprisingly found that Fgl2^{Tg} Treg are potent suppressors of alloimmune responses and that FGL2-expressing Treg promote immune tolerance.

Accordingly, the present disclosure provides a transgenic mouse whose genome comprises a recombinant fgl2 transgene, wherein the mouse ubiquitously expresses FGL2. In one embodiment, the transgene is operably connected to a promoter that drives high levels of gene expression. In another embodiment, the fgl2 transgene encodes a FGL2 protein that lacks prothrombinase activity. In a further embodiment, the fgl2 transgene comprises a nucleic acid sequence with at least 80%, 85%, 90%, 95%, 96%, 97%, 98%, 99% or 100% sequence identity to SEQ ID NO:1 and the promoter is a CAG promoter. In an additional embodiment, the mouse is humanized.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows that rFGL2 prevents rejection of fully mismatched cardiac allografts. FIG. 1A shows the determination of the plasma half-life of rFGL2. 20 μg rFGL2 was injected intravenously into fgl2^{+/+} mice and blood samples were collected at 0.25, 0.5, 1, 2, 4, 8, 16, 24, and 48 hours post-injection. Plasma levels of FGL2 are plotted against time. (n=3/time point). rFGL2 was generated in a mammalian Chinese hamster ovary (CHO) cell system. An IgG2a Fc tag was inserted at the amino terminus of murine FGL2 to improve protein solubility and stability. The Fc tag was mutated to prevent complement dependent and antibody-dependent cell-mediated cytotoxicity effects (44). A mouse IgGK chain signal peptide was inserted 5′ of the Fc tag. FIG. 1B shows the treatment schedule for rFGL2. BALB/c cardiac grafts were transplanted into fgl2^{+/+} (C57BL/6) recipients on day 0. Fgl2^{+/+} recipients were treated with 20 μg of rFGL2 intravenously on the days indicated with arrows. FIG. 1C shows the survival of cardiac allografts in rFGL2-treated and Fc-treated recipients. Graft function was measured as described in the Materials and Methods, below. (n=7 mice/group, *** p<0.001). FIG. 1 D shows the histology of cardiac allografts from rFGL2-treated and Fc-treated recipients collected at post-operative day (POD) 7. A control, non-transplanted heart is shown for comparison. Panels show hematoxylin and eosin (H&E) (original magnification 100×) or Martius Scarlet Blue (MSB) (original magnification 100×) stains. In the MSB stain, fibrinogen stains red, red blood cells stain yellow and collagen stains blue. Representative allograft from rFGL2-treated mice shows near-normal histology with minor lymphocytic infiltrate. Fc-treated grafts shows dense infiltration of mononuclear cells (arrow) with vasculitis, cardiomyocyte necrosis and associated fibrosis (dashed arrow), and fibrin deposition (arrowhead).

FIG. 2 shows the generation of fgl2^{Tg} mice. FIG. 2A shows the generation of the fgl2^{Tg} mouse using the iZIEG targeting vector (30). Prior to Cre-mediated recombination (fgl2^{LoxP} ), the first reporter gene, β-geo (LacZ+neoR), is expressed from the CAG promoter. Following Cre-mediated excision (fgl2^{Tg} ), the β-geo gene is excised and the fgl2 and enhanced green fluorescent protein (egfp) genes are brought under the control of the CAG promoter. Fgl2^{Tg} mice were generated by crossing fgl2^{LoxP} mice with Ella-cre mice, which express Cre ubiquitously, resulting in widespread FGL2 and EGFP expression. FIG. 2B shows plasma levels of FGL2 in fgl2^{Tg} mice. Heparinized blood was collected from fgl2^{+/+}, fgl2^{-/-} and fgl2^{Tg} mice and FGL2 plasma levels were measured by ELISA. mice/group, ***p<0,001). FIG. 2C shows EGFP expression in whole mount organs harvested from fgl2^{Tg} mice. The same tissues from fgl2^{-/-} mice are shown alongside the transgenic tissues for comparison. Bar shows scale for comparison. FIG. 2D shows immunofluorescence staining of cardiac tissue isolated from fgl2^{Tg} and fgl2^{-/-} mice. FGL2 protein was detected with a polyclonal rabbit antibody and is shown in red. Nuclei were counterstained with 4′,6-diamidino-2-phenylindole (DAPI) (blue). Original magnification 400×. FIG. 2E shows a western blot of cardiac tissue lysates from fgl2^{Tg} mice. FGL2 was detected using a polyclonal rabbit antibody (Veritas) and goat anti-rabbit secondary (GE Healthcare). EGFP was detected using anti-EGFP (JL-8 clone, Clontech) and a sheep anti-mouse horseradish peroxidase (HRP) conjugated secondary antibody (GE Healthcare). β-actin was detected with a mouse monoclonal antibody (clone AC15, Sigma Aldrich) and a sheep anti-mouse HRP-conjugated antibody (GE Healthcare). As a positive control, CHO cells were transiently transfected with the egfp gene. FIG. 2F shows the proportion of EGFP+ cells in total splenic mononuclear cells (SMNC), CD4⁺, CD8⁺ and CD4⁺CD25⁺ T cells populations from fgl2^{Tg} mice as detected by flow cytometry. FIG. 2G shows FGL2 production by SMNC from fgl2^{Tg} mice. SMNC isolated from fgl2^{Tg}, fgl2⁺¹⁺or fgl2^{-/-} mice were stimulated with 100 ng/ml IFN-γ (R&D Systems) or 2.5 μg/ml Concanavalin A (conA) (Sigma Aldrich). After 48 h, FGL2 levels were measured in the supernatant of unstimulated and stimulated cells, (n=3 mice/group, *p<0.05, **p<0.01, ***p<0.001).

FIG. 3 shows alterations in immune function in fgl2^{Tg} mice. FIG. 3A shows SMNC populations in fgI2^{+/+}, fgl2^{-/-} and fgl2^{Tg} mice. FIG. 3A (i) shows CD3⁺ and CD19⁺ cells and (ii) shows CD4⁺ and CD8⁺ cells as a percentage of total SMNC. (n≦3 mice/group). FIG. 3B shows T cell proliferation assays. FIG. 3B (i) shows 2.0×10⁵ CD4⁺ cells from fgl2^{+/+} or fgl2^{Tg} mice that were stimulated with irradiated 4.0×10⁵ allogeneic BALB/c SMNC and cultured for 3 days. Proliferation was measured by ³H-thymidine incorporation. (n>3 mice/group). FIG. 3B (ii) shows 1×10⁵ SMNC from fgl2^{+/+}, fgl2^{-/-} and fgl2^{Tg} mice that were stained with proliferation dye (eFluor670) and cultured for 3 days in the presence of anti-CD28 antibody (0.5 μg/ml) on an anti-CD3 antibody (1 μg/ml) coated plate. The percentage of proliferating CD4⁺ cells was determined by flow cytometry. (n=3 mice/group). FIG. 3B (iii) shows representative flow plots of CD4⁺ proliferation for fgi2^{+/+}, fgl2^{-/-} and fgl2^{Tg} SMNC. FIG. 3C shows a characterization of bone marrow-derived dendritic cells (BMDC) from fgl2^{Tg} mice. FIG. 3C (i) shows numbers of BMDC isolated from fgl2⁺¹⁺, fgl2^{-/-} or fgl2^{Tg} mice after culturing 1×10⁶ bone marrow cells for 7 days in the presence 20 ng/ml GM-CSF. FIG. 3C (ii) shows 1L-10 production by BMDC. BMDC from fgl2^{+/+}, fgl2^{-/-} and fgl2^{Tg} mice were cultured in the presence of 20 ng/ml GM-CSF for 7 days, Cells were stimulated with 20 ng/ml LPS and supernatants were collected after 48-h. IL-10 levels in supernatant were assessed by ELISA. (n=6-8 mice/group). FIG. 3D shows the characterization of Treg from fgl2^{Tg} mice. FIG. 3D (i) shows CD4⁺Foxp3⁺ T cells in SMNC from fgl2^{Tg}, fgl2^{+/+} and fgl2^{-/-} mice as a proportion of total CD4⁺ T cells, FIG. 3D (ii) shows FGL2 production by fgl2^{Tg}, fgl2^{+/+} and fgl2^{-/-} Treg activated by anti-CD3 (5 μg/ml) and IL-2 (200 U/ml, BioLegend) after 9 days. Data is presented as FGL2 (ng)/10⁶ cells. (n=3 mice/group). FIG. 3E shows a Treg suppression assay, The suppressive activity of fgl2^{Tg} and fgl2^{+/+} CD4⁺CD25⁺ regulatory T cells were compared in a standard suppression assay. 4.0×10⁴ CD4⁺CD25⁻ T cells (fgl2^{+/+}) were cultured in the presence of 0.2×10⁶ accessory cells (CD4⁻CD25⁻ SMNC, fgl2^{+/+}), 1 μg/ml ConA and titrated numbers of Treg isolated from either fgl2^{+/+} or fgl2^{Tg} mice for 3 days. Proliferation is presented as a percentage of cell inhibition compared to the proliferation of CD4⁺CD25⁻ T cells. Data were generated from five independent experiments. (*p<0.05, **p<0.01, ***p<0.001).

FIG. 4 shows that fgl2^{Tg} Treg are more potent suppressors of alloimmune responses than fgl2^{+/+} Treg. FIG. 4A shows fgl2^{Tg} Treg suppression of mixed lymphocyte reaction (MLR). 2.0×10⁵ fgl2^{+/+}. CD4⁺CD25⁻ T cells were incubated with 4.0×10⁵ irradiated BALB/c SMNC (20 cGy) and titrated numbers of either fgl2^{+/+} or fgl2^{Tg} Treg for 3 days. Proliferation was measured by ³H-Thymidine incorporation. Data is representative of 3 independent experiments (*p<0.05, **p<0.01). FIG. 4B shows Treg suppression of alloimmune responses in vivo. Rag1^{-/-} mice were reconstituted with 1.0×10⁵ CD4⁺CD25⁻ T cells (fgl2^{+/+} ) and 1.0×10⁵ fgl2^{+/+} or fgl2^{Tg} CD4⁺CD25⁺ Treg (n=12 and n=11 respectively). Control mice received T cells alone (n=3) and the sham treated group received Hank's Buffered Salt Solution (HBSS) alone. Rag1^{-/-} mice received a BALB/cJ skin graft the following day and were monitored for rejection. († p=0.07, fgl2^{Tg} vs. fg2^{+/+} Treg).

FIG. 5 shows that fgl2^{Tg} mice accept fully-mismatched allografts in the absence of immunosuppression. FIG. 5A shows graft survival in syngeneic recipients (n=3), fgl2^{+/+} recipients (n=7), fgl2^{-/-} recipients (n=8), fgl2^{Tg} recipients (n=10), BALB/c mice receiving fgl2^{Tg} grafts (n=4). Cessation of beating was associated with graft rejection and was confirmed by direct visual examination. Allografts accepted long-term in fgl2^{Tg} mice are termed tolerant (Tol). FIG. 5B shows the histology of grafts. Representative H&E staining of syngeneic (fgl2^{+/+}→fgl2^{+/+}), rejecting (BALB/c fgl2^{+/+}, BALB/c→fgl2^{-/-}, and fgl2^{Tg}→BALB/c) and tolerant (Tol) (BALB/c→fgl2^{Tg} ) grafts. Rejected grafts were lost due to acute cellular rejection characterized by vasculitis (dashed arrow), heavy lymphocytic infiltration (thick arrows) and areas of cardiomyocyte necrosis (*). Syngeneic grafts were near normal with no evidence of vasculitis and tolerant grafts showed a mild cellular infiltrate (thin arrow) but no myocyte necrosis or vasculitis. Original magnification 200×, FIG. 5C shows immunostaining for CD3⁺ and forkhead box P3 (Foxp3⁺) T cells in cardiac grafts from syngeneic , BALB/c→fgl2^{+/+}, and tolerant (Tol) BALB/c→fgl2^{Tg} recipients, Original magnification 200×. FIG. 5D shows the morphometric analysis of CD3⁺ and Foxp3⁺ cells in grafts. Graphs show (i) the absolute number of CD3⁺ cells per unit of area, (ii) the absolute number of Foxp3⁺ cells per unit of area, and (iii) the ratio of Foxp3⁺ to CD3⁺ cells in the grafts. Graphs include data from BALB/c→fgl2^{+/+}, BALB/c→fgl2^{-/-}, rejected (Rej) BALB/c→fgl2^{Tg}, and tolerant (Tol) BALB/c→fgl2^{Tg} grafts (n≧3 mice per group). FIG. 5E shows MLR responses in tolerant (Tol) fgl2^{Tg} recipients. SMNC were isolated from non-transplanted fgl2^{+/+} mice, non-transplanted fgl2^{Tg} mice, and tolerant fgl2^{Tg} recipients and stimulated with irradiated BALB/c, fgl2^{+/+} or CBA (third party) stimulators at a 1:2 ratio respectively. Cells were cultured for 3 days and proliferation was measured by ³H-thymidine incorporation over 18 hours. (*p<0.05, **p<0.01, ***p<0.001).

FIG. 6 shows that prothrombinase activity is inhibited in fgl2^{Tg} mice. To eliminate the contribution of FGL2 prothrombinase activity, the prothrombinase active site of the fgl2 cDNA in the fgl2^{Tg} transgene was mutated in order to alter the FGL2 prothrombinase active site from Ser to Ala. Prothrombinase activity was measured as the change in absorbance following the addition of a chromogenic thrombin substrate. In brief, 3×10⁵ macrophages thioglycolate-primed fgl2^{+/+}, fgl2^{-/-} and fgl2^{Tg} mice were adhered to the plate for 4 hours at 37° C., 5% CO2 in Dulbecco's modified Eagle's medium (DMEM)-10. 500 ng of murine prothrombin (Genway Biotech) was applied to the cells in 20 μL for 20 minutes. 125 μL of iced cold assay buffer (50 mM Tris, 227 mM NaCl, 1% BSA, pH 8.3) was added after to terminate the prothrombinase reaction. Supernatants were assessed for thrombin activity by the addition of 15 μL of the chromogenic thrombin substrate, Chromozym Th (Roche Applied Science), The absorbance value was measured at 420 nm every 10 minutes for 4 hours. The rate of absorbance change (Abs420 nm)/hour) was measured every 10 minutes for 4 hours, n≧3 mice per group; *p<0,05, **p<0.01.

FIG. 7A shows the cDNA sequence (referred to herein as SEQ ID NO:1) of the fgl2 gene insertion in the FGL2 transgenic mouse. Prothrombinase activity in the encoded FGL2 protein was abolished by mutating the serine at position 89 into an alanine. FIG. 7B shows the Fgl2^{S89A} construct on the Z/EG expression vector. SEQ ID NO:1 was inserted into the ZIEG vector at the site indicated by “Fgl2 S89A”. The Z/EG vector has a chicken b-actin promoter with an upstream cytomegalo virus-1E (CMV-1E) enhancer element to direct expression. Downstream of the promoter is a loxP flanked lacZ and neomycin-resistance fusion gene and three polyadenylation sites. Also provided on the vector is an enhanced green fluorescence protein (EGFP) gene and a B-globin PA signal. The lacZ gene acts as a first reporter and the EGFP gene acts as a second reporter. This vector provides lacZ and neomycin-resistance gene expression before Cre excision and EGFP expression after Cre excision.

### DETAILED DESCRIPTION

### (I) DEFINITIONS

Unless otherwise indicated, the definitions and embodiments described in this and other sections are intended to be applicable to all embodiments and aspects of the present disclosure herein described for which they are suitable as would be understood by a person skilled in the art.

The term “administering” as used herein refers to any conventional route for administering an agent(s) to a subject for use as is known to one skilled in the art. This may include, for example, administration via the parenteral (i.e. subcutaneous, intradermal, intramuscular, etc.), mucosal surface or oral route.

The term “allograft” as used herein refers to a transplant of an organ or tissue from two genetically non-identical members of the same species.

The term “analog” as used herein refers to a protein that has been modified as compared to the sequence of a protein described herein. Modifications may include, but are not limited to, amino acid substitutions, insertions, and/or deletions. The amino acid substitutions may be of a conserved or non-conserved nature. Analogs of a protein disclosed herein may be prepared by introducing mutations in the nucleotide sequence encoding the protein.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “conservative amino acid substitution” as used herein refers to replacing one or more amino acids of a protein disclosed herein with amino acids of similar charge, size, and/or hydrophobicity characteristics. Conservative substitutions are described in the patent literature, as for example, in U.S. Pat. No. 5,264,558. When only conserved substitutions are made, the resulting protein should be functionally equivalent. Non-conserved substitutions involve replacing one or more amino acids of the amino acid sequence with one or more amino acids which possess dissimilar charge, size, and/or hydrophobicity characteristics.

The terms “effective amount”, “therapeutically effective amount” and “sufficient amount” of an agent or composition of the present application is a quantity sufficient to, when administered to a subject, including a mammal, for example a human, effect beneficial or desired results, including clinical results, and, as such, a “therapeutically effective amount” or synonym thereto depends upon the context in which it is being applied. Administration of a “therapeutically effective amount” of the Treg or composition disclosed herein is defined as an amount that is sufficient to induce tolerance in a subject.

The term “FGL2”, “fibroleukin” or “fibrinogen-like 2” as used herein refers to a non-membrane bound immunomodulatory protein that is secreted by Treg immune cells_{;} including FGL2 from any species or source and including isoforms, analogs, variants or functional derivatives of such a FGL2 protein. The term also includes sequences that have been modified from any of the known published sequences of FGL2 proteins. Such modifications may result in proteins with altered function. For example, one such modification disclosed herein includes altering the nucleic acid sequence of the fgl2 gene so as to modify the site of prothrombinase activity in the encoded FGL2 protein such that prothrombinase activity is abolished. Also disclosed herein are variants such as SEQ ID NO:1 that result in a modified FGL2 protein wherein the serine at position 89 is modified in order to abolish prothrombinase activity. In one embodiment_{;} the serine is mutated into an alanine. The FGL2 protein may have any of the known published sequences for fgl2 which can be obtained from public sources such as GenBank. Examples of such sequences include, but are not limited to Accession Nos. AAL68855; P12804; 014314; NP032039; AAG42269; AAD10825; AAB88815; AAB88814; NP006673; AAC16422; AAB92553. The fgl2 sequences can also be found in WO 98/51335 (published Nov. 19, 1998) and in Marazzi et al. (1998), Ruegg et al. (1995) and Yuwaraj et al. (2001)). The aforementioned sequences are incorporated herein by reference. The FGL2 protein can be obtained from any species, optionally a mammal including human and mouse.

The term “fgl2^{Tg} mouse” as used herein refers to a mouse that has been engineered to overexpress a fgl2 transgene.

The term “FGL2 that lacks prothrombinase activity” refers to a FGL2 protein encoded by a fgl2 gene that comprises a nucleic acid sequence that been modified such that the site of prothrombinase activity of the FGL2 protein is altered in order to eliminate prothrombinase acivity.

The term “humanized” as used herein refers to a mouse that has been engrafted with human immune cells and tissues. There are many humanized mice with varying features that are known in the art and are readily available including, for example, CD34⁺ humanized mice or PMBC humanized mice available from the Jackson Laboratory (Bar Harbor, Maine). The term “humanized” as used herein with reference to the recombinant fgl2 transgene refers to a humanized mouse that has been engineered to ubiquitously express or overexpress FGL2 protein or a modified form thereof.

The term “inducing tolerance” as used herein refers to suppression or reduction of the function or activity of the immune system, or a state of non-reactivity or unresponsiveness of the immune system, to substances that have the capacity to elicit an immune response. Such substances may include organs or tissues or allergens, and would be known to one skilled in the art. The term “suppress” or “reduce” or “inhibit” a function or activity, such as an immune response, as used herein means to suppress or reduce the function or activity when compared to otherwise same conditions in the absence of Treg that overexpress FGL2.

The term “isoform” as used herein refers to a protein that contains the same number and kinds of amino acids as a protein disclosed herein, but the isoform has a different molecular structure.

The term “modified” or “modification” as used herein with reference to a fgl2 nucleic acid sequence refers to substitutions, additions, or deletions of the nucleic acid sequence which may result in isoforms, analogs, variants or functional derivatives of a FGL2 protein. Methods and techniques for mutating nucleic acid sequences are known to a person skilled in the art. Commercially available kits, such as the QuikChange XL site-directed mutagenesis kit, (Stratagene, La Jolla, Calif.) may also be used.

The term “modified” or “modification” as used herein with reference to a FGL2 protein refers to substitutions, additions, deletions or chemical equivalents of the amino acid sequence of a FGL2 protein which may result in isoforms, analogs, variants or functional derivatives of said FGL2 protein.

The term “nucleic acid sequence” refers to a sequence of nucleotide or nucleoside monomers consisting of naturally occurring bases, sugars and intersugar (backbone) linkages. The term also includes modified or substituted sequences comprising non-naturally occurring monomers or portions thereof, which function similarly. The nucleic acid sequences of the present disclosure may be ribonucleic (RNA) or deoxyribonucleic acids (DNA) and may contain naturally occurring bases including adenine, guanine, cytosine, thymidine and uracil. The nucleic acid sequences may contain naturally occurring bases including adenine, guanine, cytosine, thymidine and uracil. The sequences may also contain modified bases such as xanthine, hypoxanthine, 2-aminoadenine, 6-methyl, 2-propyl, and other alkyl adenines, 5-halo uracil, 5-halo cytosine, 6-aza uracil, 6-aza cytosine and 6-aza thymine, pseudo uracil, 4-thiouracil, 8-halo adenine, 8-amino adenine, 8-thiol adenine, 8-thio-alkyl adenines, 8-hydroxyl adenine and other 8-substituted adenines, 8-halo guanines, 8-amino guanine, 8-thiol guanine, 8-thioalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

The term “operably connected to a promoter” as used herein refers to a nucleic acid sequence that is located downstream of a promoter to form an expression cassette.

The term “overexpressed”, “increased”, “upregulated” or “elevated” as used herein in relation to FGL2 protein refers to levels of secreted FGL2 in the Treg that exceed levels of FGL2 in a Treg control cell. The expression “Treg control cell” refers to a Treg that has been obtained from an organism that has not been modified.

For example, a Treg control cell could include Treg obtained from fgl2^{+/+} mice.

The term “pharmaceutically acceptable” means compatible with the treatment of subjects, for example, mammals such as humans.

The term “promoter” as used herein refers to a nucleic acid sequence that regulates gene expression and is typically found upstream from the coding sequence in both prokaryotic and eukaryotic cells. A promoter may be characterized as strong or weak, or inducible. A strong promoter is able to drive high levels of gene expression. In contrast, a weak promoter provides only for a low level of gene expression. An inducible promoter is a promoter that is able to turn on and off of gene expression in response to an agent or stimuli. Promoters that drive high levels of gene expression that would be suitable in the context of the present disclosure would be known to one skilled in the art and include, for example, the CAG promoter. A promoter sequence may also include a regulatory sequence such as an enhancer sequence that modulates gene expression.

The term “recombinant fgl2 transgene” as used herein refers to a nucleic acid sequence encoding a FGL2 protein including sequences that have been modified from any of the known published sequences of fgl2 genes. Such modifications may result in sequences with substantial sequence homology to a known fgl2 genetic sequence. Additionally, these modifications may alter a fgl2 nucleic acid sequence so as to vary the function of the encoded FGL2 protein. For example, one such modification in the fgl2 gene disclosed herein includes altering the nucleic acid sequence so as to modify the site of prothrombinase activity in the FGL2 protein such that prothrombinase activity is abolished. Genetic variations of recombinant fgl2 transgenes are also contemplated in the present disclosure. For example, the recombinant fgl2 transgene disclosed herein may comprise at least 80%, 85%, 90%, 95%, 96%, 97%, 98%, 99% or 100% sequence identity to a known fgl2 genetic sequence or a modified version thereof. In another embodiment, the recombinant fgl2 transgene may comprise at least 80%, 85%, 90%, 95%, 96%, 97%, 98%, 99% or 100% sequence identity to SEQ ID NO:1, Also disclosed herein are variants of fgl2 such as SEQ ID NO:1 wherein the serine at position 89 of the encoded FGL2 protein is modified in order to abolish prothrombinase activity, In one embodiment, the serine is mutated into an alanine. Published sequences for fgl2 genes can be obtained from public sources such as GenBank. Examples of such sequences include, but are not limited to, Accession Nos. NM_{——}006682.2: NM_008013.4; AH005717.1; AF468959; AF104014; AF104014.2; AF104015; AF104015.2; AF025817; AF025817.1; AF025818; AF025818.1; AF036762; and AF036762.1. The aforementioned sequences are incorporated herein by reference. The fgl2 gene can be obtained from any species, preferably a mammal including human and mouse.

The term “regulatory sequence” as used herein refers to a segment of a nucleic acid molecule which is capable of altering the expression of specific genes when operatively linked to said genes. Examples of such regulatory sequences include transcriptional promoters and enhancers, a RNA polymerase binding sequence, and a ribosomal binding sequence including a translation initiation signal. Suitable promoters that may be used in the context of the disclosure include those that drive high levels of gene expression, such as the CAG promoter. Additionally, other sequences, such as an origin of replication, additional DNA restriction sites, enhancers, and sequences conferring inducibility of transcription may also be used.

The term “selectable marker” as used herein refers to a gene that facilitates the selection of host cells transformed or transfected with a recombinant molecule disclosed herein. Examples of selectable marker genes include β-galactosidase, chloramphenicol acetyltransferase, firefly luciferase, an immunoglobulin or portion thereof such as the Fc portion of an immunoglobulin preferably IgG or other genes that encode proteins which confer resistance to certain drugs such as G418 or hygromycin.

The term “sequence identity” as used herein refers to the extent to which two optimally aligned nucleic acid sequences are identical. Percent sequence identity between two sequences is determined by comparing a position in the first sequence with a corresponding position in the second sequence. When the compared positions are occupied by the same nucleotide the two sequences are conserved at that position. The degree of conservation between two sequences is often expressed as a percentage representing the ratio of the number of matching positions in the two sequences to the total number of positions compared. Sequence identity can be determined according to sequence alignment methods known in the art. Examples of these methods include computational methods such as those that make use of the BLAST algorithm, available online from the National Center for Biotechnology Information. Sequence identity is most preferably assessed by the algorithm of BLAST version 2.1 advanced search. BLAST is a series of programs that are available, for example, online from the National Institutes of Health. References to BLAST searches are: Altschul, S. F., Gish, W., Miller, W., Myers, E. W. & Lipman, D. J. (1990) “Basic local alignment search tool.” J. Mol. Biol. 215:403410; Gish, W. & States, D. J. (1993) “Identification of protein coding regions by database similarity search.” Nature Genet, 3:266272; Madden, T. L., Tatusov, R. L. & Zhang, J. (1996) “Applications of network BLAST server” Meth. Enzymol. 266:131_141; Altschul, S. F., Madden, T. L., Schaffer, A. A., Zhang, J., Zhang, Z., Miller, W, & Lipman, D. J. (1997) “Gapped BLAST and PSI_BLAST: a new generation of protein database search programs,” Nucleic Acids Res. 25:33893402; Zhang, J. & Madden, T. L. (1997) “PowerBLAST: A new network BLAST application for interactive or automated sequence analysis and annotation,” Genome Res. 7:649656.

The term “sequence with substantial sequence homology” as used herein refers to those nucleic acid sequences which have slight or inconsequential sequence variations such that the sequences function in substantially the same manner, encoding a FGL2 protein that is capable of inducing tolerance. The variations may be attributable to local mutations or structural modifications,

The term “subject” as used herein refers to any member of the animal kingdom, including mammalian kingdom such as a human.

The term “T cells” as used herein refers to thymocytes cells or T lymphocytes which are a type of lymphocytes that play a role in cell mediated immunity.

The term “transplant” or synonyms used herein refer to a medical procedure in which an organ or tissue is moved from one body to another and includes xenografts and allografts from either a living or cadaveric source.

The term “treating” or “treatment” as used herein means administering to a subject a therapeutically effective amount of the cells or compositions of the present disclosure and may consist of a single administration, or alternatively comprise a series of applications,

As used herein, and as well understood in the art, “treatment” or “treating” is also an approach for obtaining beneficial or desired results, including clinical results, Beneficial or desired clinical results can include, but are not limited to, prevention of transplant rejection, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” or “treating” as used herein can also include prolonging survival as compared to expected survival if not receiving treatment.

The term “Treg” or “regulatory T cells” as used herein refers to a sub-population of T cells which modulate the immune system and function to maintain tolerance to self-antigens and prevent autoimmune disease. A Treg of the present disclosure may comprise a recombinant fgl2 transgene disclosed herein that encodes for a FGL2 protein. The recombinant fgl2 transgene may be incorporated into a Treg using standard techniques that would be known to one skilled in the art including through the use of expression vectors or integration into the Treg genome. The recombinant fgl2 transgene may also be operably connected to a promoter that drives high levels of gene expression in the cell or other regulatory elements including transcriptional enhancers or RNA polymerase binding sequences, or a ribosomal binding sequence, including translation initiation signals. The transgene may also be modified to encode a FGL2 protein that lacks prothrombinase activity. “Treg” as used herein may also refer to a Treg that has been isolated from a transgenic mouse disclosed herein.

The term “variant” as used herein in the context of a nucleic acid molecule includes nucleic acid molecules comprising nucleic acid sequences having substantial sequence homology or sequence identity with a nucleic acid sequence disclosed herein, for example, a known fgl2 gene sequence or a modified version thereof, or SEQ ID NO:1 . Variants may comprise at least 80%, 85%, 90%, 95%, 96%, 97%, 98%, 99% or 100% sequence identity to a nucleic acid disclosed herein. Variants further include nucleic acid sequences which differ from a nucleic acid sequence disclosed herein due to degeneracy in the genetic code. Such nucleic acids encode functionally equivalent proteins but differ in sequence from the above mentioned sequences due to degeneracy in the genetic code.

The term “variant” as used herein in the context of a protein disclosed herein includes modifications, substitutions, additions, or chemical equivalents of the amino acid sequence of the protein disclosed herein that perform substantially the same function as the protein in substantially the same way. For example, a variant of a protein disclosed herein includes, without limitation, conservative amino acid substitutions.

The term “vector” as used herein includes a recombinant vector containing a nucleic acid molecule disclosed herein operably linked to any necessary regulatory elements, and includes expression vectors that allow for the transcription and translation of the inserted sequence to ensure expression of the protein of interest. Furthermore, vectors of the present disclosure may also contain selectable marker genes which facilitate the selection of host cells transformed or transfected with a recombinant molecule disclosed herein.

The term “xenograft” as used herein refers to a transplant of an organ or tissue from one species to another.

### (II) REGULATORY T CELLS

It has been shown herein that fgl2^{Tg} Treg are potent suppressors of alloimmune responses and that FGL2-expressing Treg promote tolerance.

Accordingly, the present disclosure provides a regulatory T cell (Treg) comprising a recombinant fgl2 transgene. The fgl2 transgene comprises a nucleic acid encoding a FGL2 protein and can be any of the known sequences in the art or a modified form thereof as previously described. In one embodiment, the fgl2 transgene encodes a FGL2 protein that lacks prothrombinase activity. In a further embodiment, the fgl2 transgene comprises a nucleic acid sequence with at least 80%, 85%, 90%, 95%, 96%, 97%, 98%, 99% or 100% sequence identity to SEQ ID NO:1.

A recombinant fgl2 transgene disclosed herein may be incorporated into a Treg using standard techniques that would be known to one skilled in the art including through the use of an appropriate expression vector which ensures good expression of the FGL2 peptide in the host Treg. The Treg can be transfected with the fgl2 transgene in vitro or in vivo. Various constructs can be used to deliver nucleic acid molecules described herein. Possible expression vectors include but are not limited to cosmids, plasmids, or modified viruses (e.g. replication defective retroviruses, adenoviruses and adeno-associated viruses), so long as the vector is compatible with the Treg used. The expression vectors are suitable for transformation of a Treg, meaning that the expression vectors contain a nucleic acid molecule and regulatory sequences operatively linked to the nucleic acid molecule in a manner which allows expression of the nucleic acid in the Treg.

The disclosure therefore includes a recombinant expression vector containing a nucleic acid molecule encoding a FGL2 protein and the necessary regulatory sequences for the transcription and translation of the inserted sequence in a Treg.

Suitable regulatory sequences which may be used in the context of the present disclosure may be derived from a variety of sources, including bacterial, fungal, viral, mammalian, or insect genes (for example, see the regulatory sequences described in Goeddel, Gene Expression Technology: Methods in Enzymology 185, Academic Press, San Diego, Calif. (1990)). Selection of appropriate regulatory sequences may be readily accomplished by one of ordinary skill in the art. Examples of such regulatory sequences include transcriptional promoters and enhancers, a RNA polymerase binding sequence, and a ribosomal binding sequence including a translation initiation signal. Suitable promoters that may be used in the context of the disclosure include those that drive high levels of gene expression, such as the CAG promoter. Additionally, other sequences, such as an origin of replication, additional DNA restriction sites, enhancers, and sequences conferring inducibility of transcription may also be used.

In one embodiment, the transgene is operably connected to a promoter that drives high levels of gene expression. In another embodiment, the promoter is a CAG promoter.

The recombinant expression vectors may also contain a selectable marker gene which facilitates the selection of host cells transformed or transfected with a recombinant molecule disclosed herein. Examples of selectable marker genes include β-galactosidase, chloramphenicol acetyltransferase, firefly luciferase, an immunoglobulin or portion thereof such as the Fc portion of an immunoglobulin preferably IgG or other genes that encode proteins which confer resistance to certain drugs such as G418 or hygromycin. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance, transformant cells can be selected with G418. Cells that have incorporated the selectable marker gene will survive, while the other cells die. This makes it possible to select for expression of the vectors disclosed herein. It will be appreciated that selectable markers may be introduced on a separate vector from the nucleic acid of interest.

Recombinant expression vectors containing a recombinant fgl2 transgene can be introduced into Treg to produce a transformed Treg using techniques known in the art such as transformation or transfection. The terms “transformed with”, “transfected with”, “transformation” and “transfection” are intended to encompass introduction of a nucleic acid molecule (e.g. a vector) into a Treg by one of many possible techniques known in the art including, for example, using conventional techniques such as calcium phosphate or calcium chloride co-precipitation, ©EAE-dextran-mediated transfection, lipofectin, electroporation or microinjection. Suitable methods for transforming and transfecting host cells can be found in Sambrook et al, (Molecular Cloning: A Laboratory Manual, 2nd Edition, Cold Spring Harbor Laboratory press (1989)), and other such laboratory textbooks.

In another embodiment, the Treg is isolated from a transgenic mouse disclosed herein below.

### (III) METHODS AND USES

The ability to induce immune tolerance could assist in the treatment of various disorders or conditions where it is desirable to suppress an immune response. This may include preventing the rejection of transplanted organs or tissues and for treating graft versus host disease, autoimmune disease and allergies.

Accordingly, the present disclosure provides a method of inducing tolerance in a subject in need thereof comprising administering to the subject a therapeutically effective amount of a Treg or composition disclosed herein. The present disclosure also provides a use of a therapeutically effective amount of a Treg or composition disclosed herein for inducing tolerance in a subject in need thereof. Further provided is a use of an effective amount of a Treg or composition disclosed herein in the preparation of a medicament for inducing tolerance in a subject in need thereof. Even further provided is an effective amount of a Treg or composition disclosed herein for use in inducing tolerance in a subject in need thereof.

The subject may be a mammal, optionally a human.

Determining whether a particular Treg or composition is capable of inducing tolerance can be assessed by known in vitro immune assays including, but not limited to, inhibiting a mixed leucocyte reaction; inhibiting T-cell proliferation; inhibiting interleukin-2 production; inhibiting IFNγ production; inhibiting a Th1 cytokine profile; inducing IL-4 production; inducing TGFβ production; inducing 1L-10 production; inducing a Th2 cytokine profile; inhibiting immunoglobulin production; altering serum immunoglobulin isotype profiles (from those associated with Th1 type immunity-e.g. in the mouse, IgG1 and IgG2a, to those associated with Th2 type immunity-e.g. in the mouse, IgG2b, IgG3); inhibition of dendritic cell maturation; Elispot for antibody; flow for memory cells and markers of T cell and B cells; intracellular flow cytometry for generation of inducible Treg; and any other assay that would be known to one of skill in the art to be useful in detecting immune suppression.

The method of the disclosure may be used to treat any disease or condition where it is desirable to induce tolerance or to suppress an immune response. This may include preventing the rejection of transplanted organs or tissues and for treating graft versus host disease, autoimmune disease and allergies.

Accordingly, in one embodiment, tolerance is induced to a transplanted organ or tissue. In another embodiment, the Treg or composition is administered prior to, during or after the transplantation of the organ or tissue. The transplanted organ or tissue may be selected from the group comprising heart, kidney, liver, lung, pancreas, pancreatic islets, brain tissue, intestine, thymus, bone, tendons, cornea, skin, heart valves, nerves, veins or haematopoietic cells, In another embodiment, the transplanted tissue is skin. In an additional embodiment, the transplanted organ is a heart. The transplant may also be an allograft transplant,

In a further embodiment, tolerance is induced in order to treat graft versus host disease. As used herein, “graft versus host disease” refers to a disease wherein the immune cells of a transplant mount an immune attack on the recipient's immune system. This can occur when the tissue to be transplanted contains immune cells such as when bone marrow or lymphoid tissue is transplanted when treating leukemias, aplastic anemias and enzyme or immune deficiencies, for example. One of skill in the art can determine whether or not a Treg or composition disclosed herein is useful in treating or preventing graft versus host disease. As mentioned previously, one of skill in the art can readily test a Treg or composition disclosed herein for its ability to induce tolerance or suppress an immune response using known in vitro assays.

In an additional embodiment, tolerance is induced in order to treat an autoimmune disease. In another embodiment, the autoimmune disease is selected from the group comprising arthritis, type 1 insulin-dependent diabetes mellitus, adult respiratory distress syndrome, inflammatory bowel disease, Crohn's disease, dermatitis, meningitis, thrombotic thrombocytopenic purpura, Sjogren's syndrome, encephalitis, uveitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, tissue specific autoimmunity, degenerative autoimmunity delayed hypersensitivities, autoimmune complications of AIDS, atrophic gastritis, ankylosing spondylitis or Addison's disease.

In an autoimmune disease, the immune system of the host fails to recognize a particular antigen as “self” and an immune reaction is mounted against the host's tissues expressing the antigen. Normally, the immune system is tolerant to its own host's tissues and autoimmunity can be thought of as a breakdown in the immune tolerance system. One of skill in the art can determine whether or not a Treg or composition disclosed herein is useful in preventing or treating autoimmune disease. As mentioned previously, one of skill in the art can readily test a Treg or composition disclosed herein for its ability to induce tolerance or suppress an immune response using known in vitro assays. In addition the Treg or composition disclosed herein can also be tested for its ability to prevent autoimmunity in an animal model. Many animal models are available for such testing, including, but not limited to, experimental allergic encephalomyelitis which is an animal model for multiple sclerosis, animal models of inflammatory bowel disease (induced by immunization, or developing in cytokine-knockout mice), and models of autoimmune myocarditis and inflammatory eye disease.

In another embodiment, tolerance is induced in order to treat an allergic reaction. In an allergic reaction, a subject's immune system mounts an attack against a generally harmless, innocuous antigen or allergen. Allergies that may be prevented or treated using the methods of the disclosure include, but are not limited to, hay fever, asthma, atopic eczema as well as allergies to poison oak and ivy, house dust mites, bee pollen, nuts, shellfish, penicillin and numerous others. One of skill in the art can determine whether or not a Treg or composition disclosed herein is useful in treating or preventing an allergy. As mentioned previously, one of skill in the art can readily test a Treg or composition disclosed herein for its ability to induce tolerance or suppress an immune response using known in vitro assays.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

### (IV) TRANSGENIC MICE

The present disclosure describes the generation of FGL2 transgenic mice (fgl2^{Tg} ) that have been engineered to express a modified fgl2 transgene driven by the CAG promoter, wherein the transgene has been modified such that the prothrombinase active site of the FGL2 protein is altered from Ser to Ala in order to eliminate prothrombinase activity, fgl2^{Tg} mice ubiquitously overexpress FGL2 such that plasma levels of FGL2 were determined to be ˜7-fold higher in fgl2^{Tg} mice than in fgl2^{+/+} mice. These mice developed normally and show no evidence of the autoimmune glomerulonephritis seen in fgl2^{-/-} mice. Immune characterization shows that fgl2^{Tg} T cells are hypoproliferative to stimulation with alloantigens or anti-CD3 and anti-CD28 stimulation, and fgl2^{Tg} Treg have increased immunosuppressive activity compared with fgl2^{+/+} Treg. The data disclosed herein also show that tolerance in fgl2^{Tg} recipients involves changes in Treg and T cell activity that contribute to a higher intragraft Treg to T cell ratio and acceptance of fully mismatched allografts,

Accordingly, the present disclosure provides a transgenic mouse whose genome comprises a recombinant fgI2 transgene, wherein the mouse ubiquitously expresses FGL2. In one embodiment, the transgene is operably connected to a promoter that drives high levels of gene expression. In another embodiment, the fgl2 transgene encodes a FGL2 protein that lacks prothrombinase activity. In a further embodiment, the fgl2 transgene comprises a nucleic acid nucleic acid sequence with at least 80%, 85%, 90%, 95%, 96%, 97%, 98%, 99% or 100% sequence identity to SEQ ID NO:1 and the promoter is a CAG promoter. In an additional embodiment, the mouse is humanized. In yet another embodiment, the mouse is a fgl2^{Tg} mouse,

Techniques for the preparation of a transgenic mouse whose genome comprises a recombinant fgI2 transgene would be known to a person skilled in the art. For example, nucleic acid constructs containing a recombinant fgl2 transgene can be inserted into a vector using restriction digest sites. Exemplary vectors including iZ/EG, pUC18/NotI, Bluescript II, or pGEM7. The construct may also be flanked by loxP sites in order to allow for Cre recombinase-mediated excision of the gene, permitting the conditional control of gene expression. The recombinant vector containing a nucleic acid molecule disclosed herein may also include regulatory sequences for the transcription and translation of the inserted construct.

The nucleic acid constructs or vectors containing the construct can be used to transfect cells such as embryonic stem cells. Embryonic stem cells are useful as they can integrate into and become part of the germline of the developing embryo so as to create germline transmission of the nucleic acid construct or vector carrying the fgI2 gene or a modified version thereof. Any embryonic stem cell that can integrate into the developing embryo may be used in the present invention. The embryonic stem cell is generally of the same species as the transgenic animal to be prepared i.e., to make a transgenic mouse, mouse embryonic stem cells are used. The cells can be cultured prior to transfection with the nucleic acid construct or vector using methods well known in the art including the methods taught by Robinson in Teratocarcinomas and Embryonic Stem Cells: A Practical Approach, E. J. Robinson editor, IRL Press, Washington, D.C., 1987.

The construct or vector can be inserted into the cells using techniques known in the art including electroporation, microinjection and calcium phosphate treatment. If a vector is to be inserted, prior to insertion the vector is linearized using a suitable restriction enzyme that cuts within the vector sequence and not within the nucleic acid sequence containing the fgl2 transgene or any regulatory elements. After insertion into the cells, the nucleic acid construct integrates with the genomic DNA of the cell in order to prevent or inhibit transcription of the native fgl2 gene. Preferably, the insertion occurs by homologous recombination wherein regions of the fgl2 gene in the nucleic acid construct hybridize to the homologous fgl2 sequences in the cell and recombine to incorporate the construct into the endogenous fgl2 sequence.

After transfection, the cells are cultured under conditions to detect host cells transfected with a recombinant molecule disclosed herein. For example, a selectable marker gene may be included on the construct or vector which facilitates the selection of host cells transfected with a recombinant molecule disclosed herein. This marker gene may comprise an antibiotic resistance marker such that when the cells are cultured in that antibiotic only the transfected cells are able to replicate. In particular, when the neo-R gene is present, the cells can be cultured in the neomycin drug analog G418. The cells containing the vector survive while the non-transfected cells die. The DNA of the surviving cells can be analyzed using Southern Blot technology and/or Polymerase Chain Reaction (PCR) in order to identify the cells with the proper integration of the construct.

The embryonic stem cells containing a fgl2 nucleic acid construct or vector can then be used to prepare a transgenic mouse. In particular, the embryonic stem cells are inserted to an early embryo for example using microinjection. For microinjection, approximately 10-20 embryonic stem cells are collected into a micropipette and injected into 3-5 day old blastocysts, preferably 3½ day old blastocysts, recovered from female mice. The injected blastocysts are re-implanted into a foster mother. When the pups are born, typically 20-21 days later, they are screened for the presence of the nucleic acid construct of the invention. For example, the tail tissue of the pups may be screened using Southern blots and/or PCR. The heterozygotes are identified and can then be crossed with each other to generate homozygous transgenic animals.

The transgenic mice described herein may be useful as research tools. Accordingly, another aspect of the present disclosure relates to the use of the transgenic mouse as an in vivo model organism. In another embodiment, the mouse may be for use as an in viva model organism for studying immune related disorders. In an additional embodiment, the mouse may be for use as an in vivo model organism for studying autoimmune disorders, graft rejection, allergies and graft versus host disease.

### (V) COMPOSITIONS

The disclosure also provides a composition comprising a Treg disclosed herein and a pharmaceutically acceptable carrier. In one embodiment, the composition further comprises one or more immunosuppressive agents. The composition may be used, for example, to induce immune tolerance or to suppress an immune response.

The compositions containing Treg can be prepared by known methods for the preparation of pharmaceutically acceptable compositions which can be administered to subjects such that an effective quantity of the active agent is combined in a mixture with a pharmaceutically acceptable carrier, vehicle or diluent.

Suitable carriers, vehicles and diluents are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions comprising the Treg in association with one or more pharmaceutically acceptable vehicles, carriers or diluents, and contained in buffered solutions with a suitable pH and iso-osmotic with the physiological fluids. Compositions include, without limitation, lyophilized powders or aqueous or non-aqueous sterile injectable solutions or suspensions, which may further contain antioxidants, buffers, bacteriostats and solutes that render the compositions substantially compatible with tissue or blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The pharmaceutical composition may be supplied, for example but not by way of limitation, as a lyophilized powder which is reconstituted with sterile water or saline prior to administration to the patient.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions may be in the form of pharmaceutically acceptable salts which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol, histidine, procaine, etc.

The pharmaceutical compositions can be intended for administration to a subject such as an animal including a mammal or a human. The term “administration” or synonyms used herein refer to any conventional route for administering an agent(s) to a subject for use as is known to one skilled in the art. For example, the compositions disclosed herein may be administered by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

The dosage amount of the agent(s) and/or therapeutically effective amount will vary depending upon various factors, such as recipient needs and attributes (for example: health, age, weight and sex), the nature and extent of the symptoms, the desired effect, the frequency of the treatment, the pharmaceutical formulation and the chosen route of administration. One of skill in the art can determine the appropriate dosage based on the above factors. One skilled in the art will appreciate that the dosage regime can be determined and/or adjusted without undue experimentation in order to provide the optimum dose. The Treg or compositions may be administered initially in a suitable dosage that may be adjusted as required to provide the optimum dose, depending on the clinical response.

Immunosuppressive agents that may be used in the context of the disclosure would be known to one skilled in the art and comprise molecules that prevent the occurrence of an immune response or weaken the immune system of a subject. Such agents may reduce or prevent T cell proliferation. Exemplary immunosuppressive agents include glucocorticoids (e.g. methylprednisolone, prednisone, prednisolone, cortisone, cortisol, betamethasone, triamcinolone acetonide or beclometasone dipropionate), cytostatics/antiproliferative agents (e.g.

alkylating agents, antimetabolites, methotrexate, mycophenolic acid, mycophenolate mofetil, mycophenolate sodium or azathioprine), antibodies (e.g. IL-2 receptor-directed antibodies such as basiliximab or daclizumab, or CD3-directed antibodies such as muromonab-CD3), drugs that act on immunophilins (e.g. ciclosporin, tacrolimus or sirolimus), interferons, opioids, TNF binding proteins (e.g. infliximab, etanercept or adalimumab), mycophenolate, and small biological agents.

The following non-limiting examples are illustrative of the present disclosure:

### (VI) EXAMPLES

### Materials and Methods

### Animals

BALB/cJ, Rag1^{-/-}, CBA/J, Ella-cre (C57BL/6) (Jackson Laboratory, Bar Harbor, Me.), fgl2^{+/+} (C57BL/6J), fgl2^{-/-} and fgl2^{Tg} mice were housed at the Ontario Cancer Institute Animal Resource Centre (Toronto, Canada) in specific pathogen free conditions. Animals were treated in accordance with guidelines set by the Canadian Council for Animal Care, University Health Network and University of Toronto. Female mice 6-12 weeks of age were used for experiments.

Production and Purification of Mouse rFGL2

Mouse rFGL2 (Fc-tagged) was produced in CHO cells and purified as previously described (19). Purity was determined by SDS-PAGE and the concentration was determined using a mouse FGL2 ELISA kit (BioLegend, San Diego, Calif.).

### Heterotopic Cardiac Transplantation

Intra-abdominal heterotopic cardiac transplants were performed as previously described (29). Donor hearts were harvested from 4-6 week old mice and inserted into the abdomen of recipient mice. Rejection was defined as the complete cessation of heart contractions and was confirmed by direct visual examination.

Generation of fgl2^{Tg} Mice

The cDNA of the mouse fgl2 gene was obtained from C57BL/6 heart RNA. Bgl II and XhoI sequences were added to the 5′ and 3′ ends of the fgl2 cDNA sequence. A Bgl II internal restriction site was removed and the active site of fgl2 prothrombinase activity was mutated from Ser to Ala using the QuikChange XL site-directed mutagenesis kit (Stratagene, La Jolla, CA). A KOZAK sequence was added 5′ to the fgl2 start codon. This fgl2 cDNA was inserted into the iZ/EG vector (iZiEG-fgl2^{Tg}) (30). Fgl2^{Tg} mice were generated by the aggregation of ES cells and tetraploid embryos as previously described (30, 31). R1 ES cell- iZ/EG-fgl2^{Tg} clones were selected for single copy insertion and strong expression of the β-gal gene. Clone 3C3 yielded viable offspring. Chimeric fgl2^{LoxP} mice were crossed with fgl2^{-/-} mice twice followed by two crosses with Ella-cre;fgl2^{-/-} mice.

β-gal Staining and EGFP Visualization

LacZ staining was performed as previously described (31). Bright field photomicrographs were taken using a MZ FLIII stereomicroscope (Leica, Switzerland). An EGFP2 filter was used for visualization of EGFP whole mount organ fluorescence.

### FGL2 Immunohistochemistry

Heart tissues were embedded in OCT (Sakura Finetek, Torrance, Calif.), snap frozen, cut in 7 μm sections, and fixed in acetone for 10 minutes at room temperature. Endogenous biotin was blocked using an Avidin/Biotin blocking kit (Vector Laboratories, Burlingame, Calif.). Sections were then blocked by addition of normal goat serum (20 μl/ml) (Vector Laboratories) followed by staining with a 1:200 dilution of a rabbit polyclonal anti-FGL2 antibody (Veritas, Canada) for 60 minutes at room temperature. The primary antibody was detected using the Vector Elite Stain ABC kit followed by incubation with Texas-Red Streptavidin (Vector Laboratories) and counterstained with DAPI (Life Technologies, Carlsbad, Calif.). Slides were visualized on an Olympus IX81 Inverted Microscope (Olympus, Tokyo, Japan).

### Western Blots

Western blots were performed as previously described (32). CHO cells transfected with egfp were used as a positive control. Samples were resolved on a 12.5% SDS-PAGE gel and transferred onto a polyvinylidene difluoride membrane (Millipore, Billerica, Mass.). Blots were developed using an ECL-based system (GE Healthcare, Little Chalfont, United Kingdom).

### Flow Cytometry

A single cell suspension of splenic mononuclear cells (SMNC) was incubated with Fc Block and was stained with CD19-PE, CD8-APC, CD4, Foxp3-PE, CD4-PC5 or -PE (eBioscience, San Diego, Calif.) or CD25-PE (Miltenyi Biotec, San Diego, Calif.). For intracellular staining, cells were treated with a fixation and permeabilization buffer (eBioscience) for 18 hours, incubated with Fc Block and stained with Foxp3-PE, Cells were visualized using a LSR II analyzer (BD Biosciences, Franklin Lakes, N.J.). Data was analyzed using FlovvJo software version 9.6 (Tree Star Inc., Ashland, Oreg.).

### Plasma FGL2 Levels

Blood was collected from the saphenous vein or by cardiac puncture using heparinized capillary tubes or heparin-coated syringes. Plasma FGL2 levels were determined using a mouse FGL2 ELISA kit (BioLegend),

### T Cell Proliferation Assays

SMNC were labelled with 10 μM eFluor670 proliferation dye and cultured in the presence of anti-CD28 antibody (0.5 μg/ml) on an anti-CD3 antibody (1 μg/ml) coated plate as per manufacturer's instructions (eBioscience). After 3 days, cells were harvested, stained for viability using eFluor 450 (eBioscience) and CD4+ expression and analyzed by flow cytometry.

### Generation of Bone Marrow-Derived Dendritic Cells (BMDC)

Bone marrow was harvested and cultured to generate BMDC as previously described (33). In brief, 10⁶ cells were incubated for 7 days in RPMI-5 media supplemented with 20 ng/ml recombinant mouse granulocyte-macrophage colony-stimulating factor (GM-CSF) (Bio Basic Inc., Amherst, N.Y.). Media was replenished every two days. Cells were harvested and cell number and viability was assessed by trypan blue exclusion. At day 7, BMDCs were harvested from cultures and stimulated with 20 ng/ml LPS (Sigma-Aldrich, Saint Louis, Mo.) for 48 hours. IL-10 levels in the supernatant were measured using a mouse 1L-10 ELISA kit (BioLegend).

### Treg Suppression Assays

Treg suppression was measured using an assay described previously (23, 34). For MACS sorting, SMNC were blocked with rat IgG (R&D Systems, Minneapolis, MN). CD4⁺CD25⁺ cells were magnetically sorted using anti-CD25 microbeads and anti-CD4 microbeads (Miltenyi Biotec). The enriched CD4^{÷}CD25⁺ T cell fraction was FACS sorted into CD4⁺CD25⁺ regulatory T cells and CD4⁺CD25⁻ T cells on a BD FACS Aria (BD bioscience). The population purity was >97%. Fgl2^{+/+} CD4⁻CD25⁻ accessory cells or BALB/c SMNC were exposed to 2000 cGy using a γ-irradiator. For the standard Treg suppression assay, cells were cultured in the presence of 1 μg/ml ConA for 3 days at the indicated ratios. For the allogeneic Treg suppression assay, cells were cultured in the presence of allogeneic BALB/c SMNC for 3 days at the indicated ratios. 1 μCu of ³H-thymidine (GE Healthcare) was then added to the culture for 18 hours to measure proliferation.

### Cellular Reconstitution of Rag1^{-/-} Mice and Skin Transplantation

CD4⁺ cells were isolated from SMNC using anti-CD4 microbeads (Miltenyi Biotec) and then sorted into CD4⁺CD25⁺ Treg and CD4⁺CD25⁻ T cells using a BD FACS Aria. Treg and T cells were injected intravenously into Rag1^{-/-} mice using an adapted protocol (35). On the following day, a full thickness skin graft from BALB/cJ mice (1 cm2 in size) was grafted onto the dorsum of the Rag1^{-/-} mice. Complete necrosis of the graft was defined as rejection.

### Histology and Immunohistochemistry

Allografts and non-transplanted hearts were processed for routine histology and immunohistochemistry following fixation with 10% formalin.

Immunoperoxidase staining was performed with rat anti-mouse CD3 and rat anti-mouse Foxp3 antibodies (eBioscience). For morphometry; stained cells were identified using the program SPECTRUM version 10.2.2.2317 (Aperio Technologies Inc., Vista, Calif.).

### Mixed Lymphocyte Reaction (MLR)

SMNC suspensions were prepared using Lympholyte-M density separation medium (Cedarlane, Burlington, Canada). Allogeneic (BALB/c, CBA/J) or syngeneic SMNC were irradiated with 2000 cGy. Proliferation was measured by the addition of 1 μCi of ³H-thymidine (GE Healthcare) 18 hours prior to the termination of the assay. Samples were harvested and events were recorded on a TopCount scintillation counter (PerkinElmer, Waltham, Mass.).

### Statistics

Statistical significance was verified using a Student's t-test, one-way or two-way ANOVA, as indicated using PRISM v5a (GraphPad Software Inc., La Jolla, Calif.). Survival data was analyzed using the Log-rank (Mantel-Cox) test and chi-square contingency tables. p values≧0.05 were considered statistically significant. Data are expressed as mean±standard deviation (SD) unless otherwise stated.

### Results

prFGL2 Prevents Rejection of Fully Mismatched Cardiac Grafts

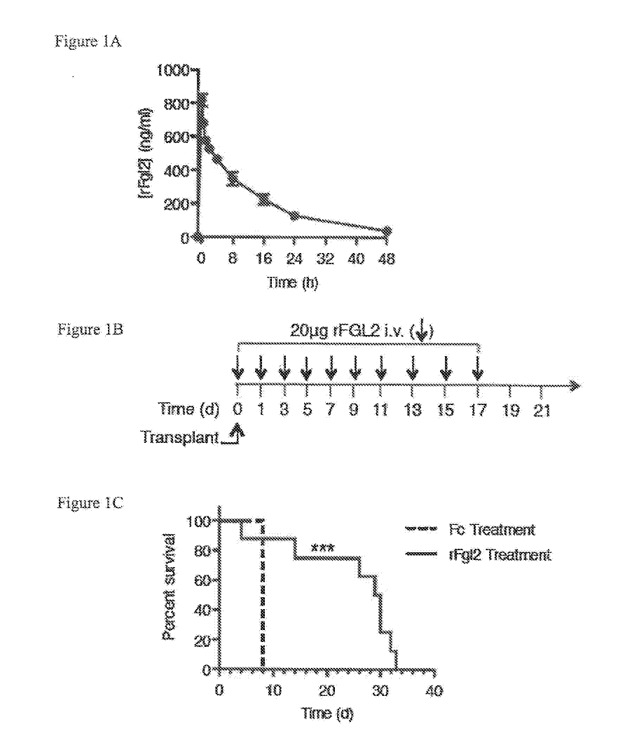

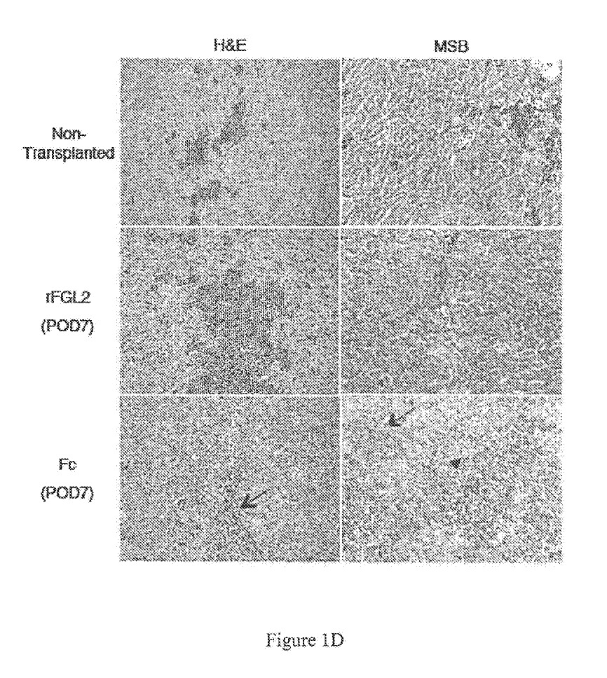

To determine the plasma half-life of rFGL2, mice were treated with 20 μg of rFGL2 intravenously and blood samples were collected pre-injection and at various time points postinjection. FGL2 plasma levels peaked within 15 minutes (816 ng/ml) and decreased to baseline by 48 hours (FIG. 1A), The half-life of rFGL2 in the plasma was determined to be 6-8 hours. Based on this finding, BALB/c (H-2^{d}) hearts were transplanted into fgl2^{+/+} (H-2^{b}) mice, and recipients were treated with rFGL2 or Fc-tag alone from day 0 to day 17 using a dosing regime as shown (FIG. 1B). All Fc-treated mice rejected their grafts by day 8 similar to untreated mice, whereas rFGL2-treated mice accepted their grafts as long as rFGL2 was administered (FIG. 1C). Following cessation of rFGL2 treatment, all allografts were rejected within 15 days. Allografts from rFGL2 treated mice had near normal histology during treatment, whereas allografts from Fc-treated mice showed a heavy infiltration of mononuclear cells with vasculitis and cardiomyocyte necrosis (FIG. 1D),

### Generation and Characterization of FGL2 Transgenic Mice

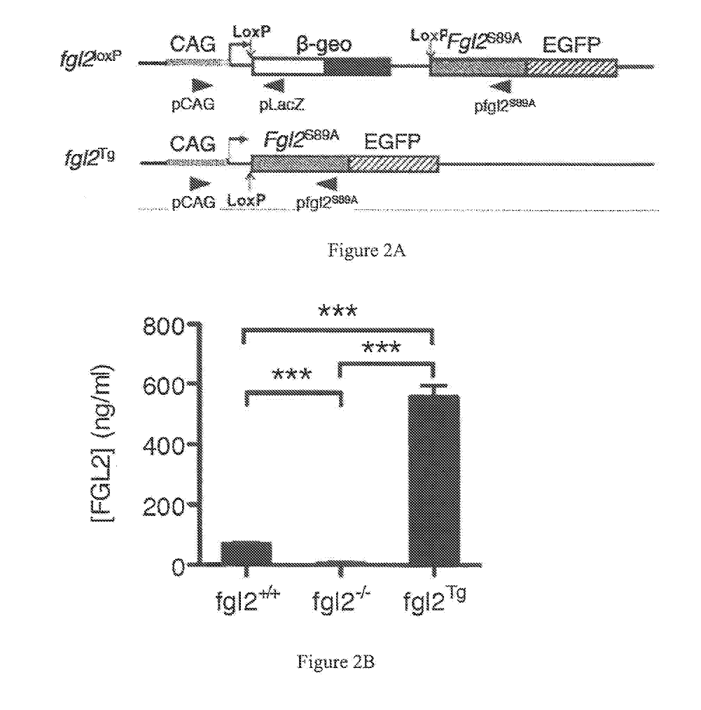

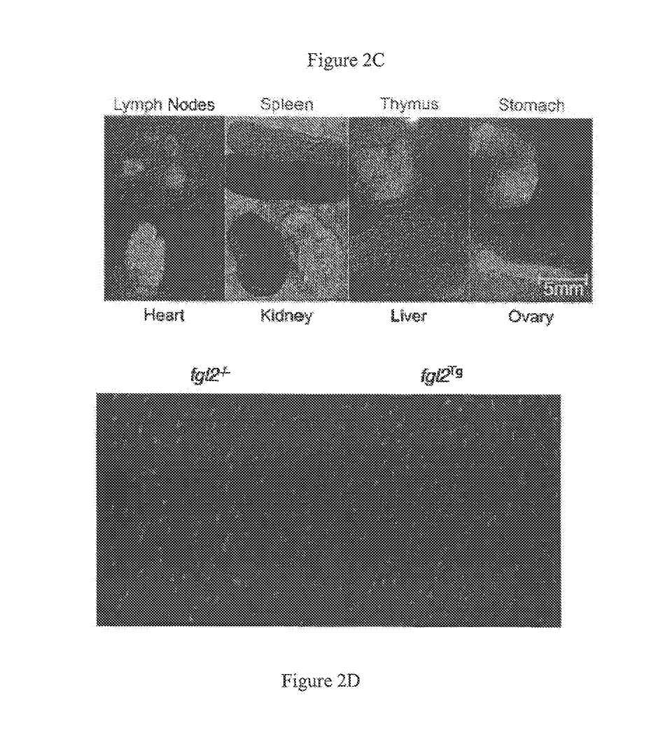

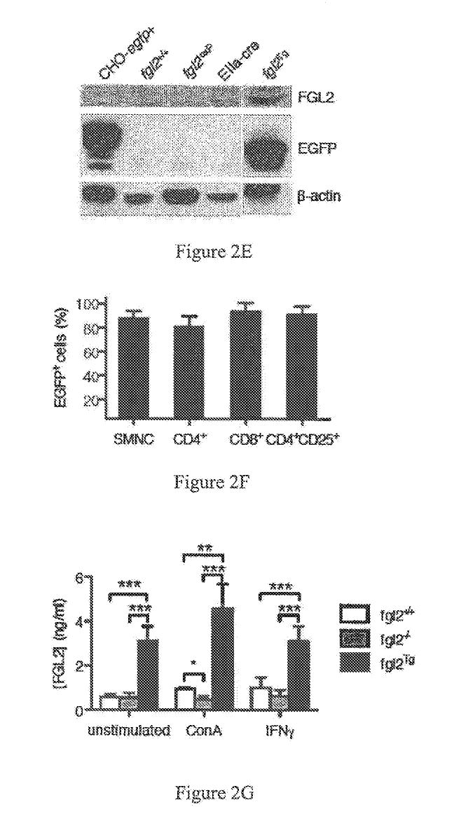

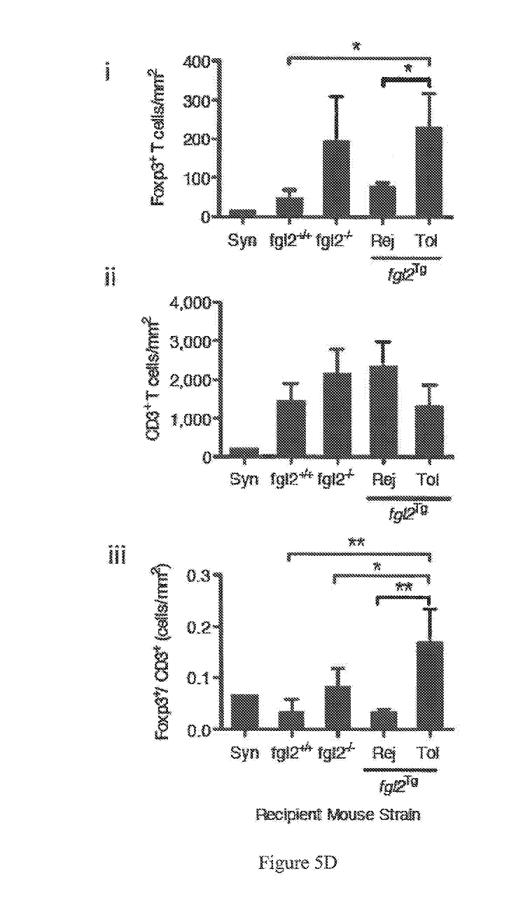

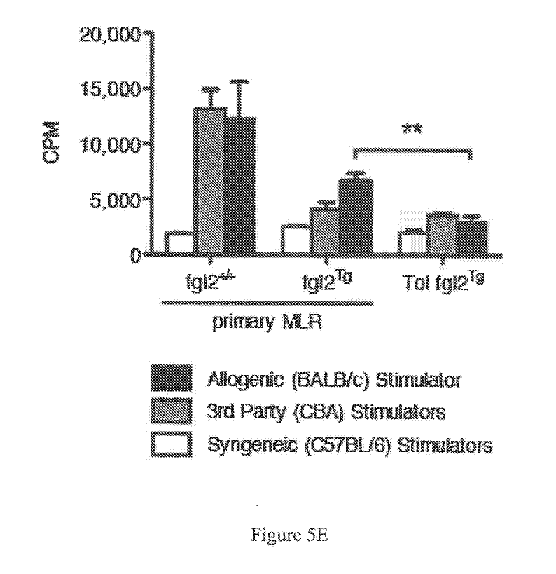

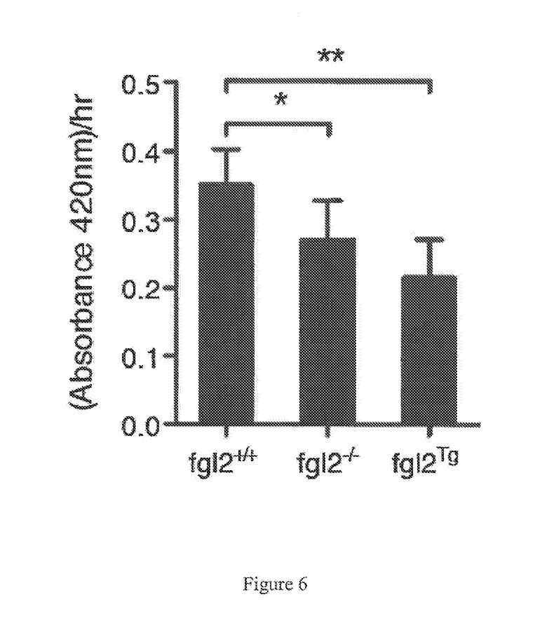

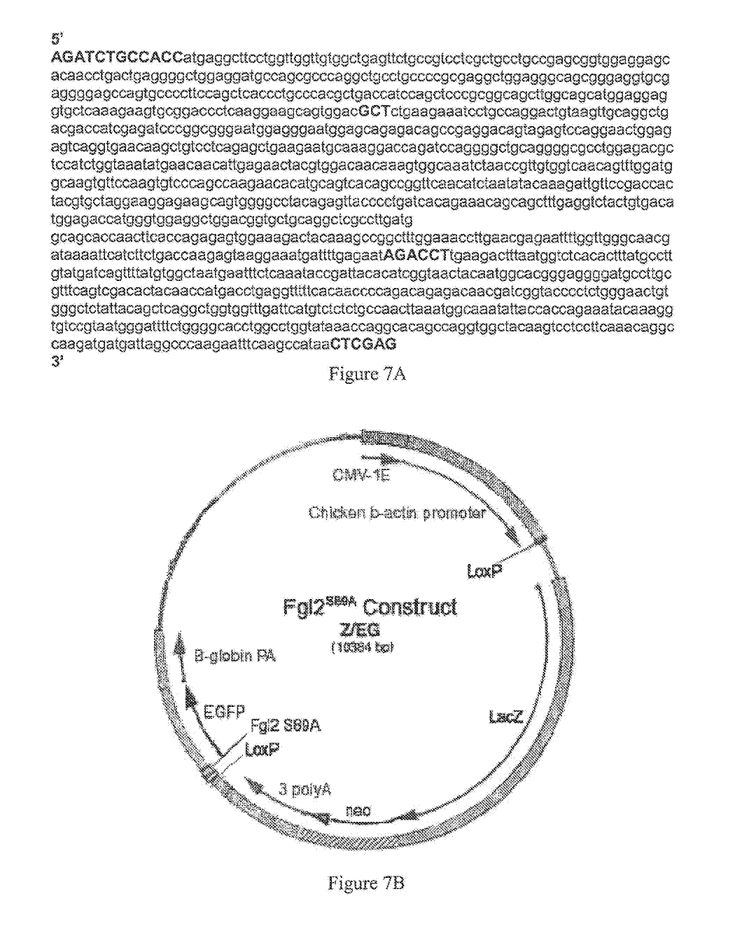

To determine if constitutive expression of FGL2 would lead to acceptance of heart grafts, fgl2^{Tg} mice in which the expression of the fgl2 transgene is driven by the CAG promoter were generated (30). Prior to Cre-mediated excision (fgl2^{LoxP}), expression of the β-geo reporter gene was controlled by the CAG promoter. Cre-mediated excision (fgl2^{Tg} ) removes the β-geo gene and brings fgl2 and egfp (second reporter) under control of the CAG promoter (FIG. 2A). The fgl2 transgene was mutated at the prothrombinase active site to eliminate prothrombinase activity. Founder fgl2^{LoxP} mice were crossed with fgl2^{-/-} mice to remove endogenous expression of FGL2. fgl2^{LoxP} mice were crossed to Ella-cre; fgl2^{-/-} mice to generate fgl2^{Tg} mice that express FGL2 and EGFP ubiquitously (36). Plasma levels of FGL2 were 7-fold higher in fgl2^{Tg} than in fgl2^{+/+} mice (559.2±115.1 ng/ml vs. 75.43±6.24 ng/ml) (FIG. 2B), whereas FGL2 was undetectable in fgl2^{-/-} mice. Adult 6-8 week old fgl2^{Tg} mice were nearly indistinguishable from fgl2^{-/-} littermates with respect to gross anatomy, standard haematological profiles and biochemical profiles (see Tables 1 and 2). Importantly, kidneys from fgl2^{Tg} mice showed no evidence of glomerulonephritis (data not shown) as was reported in fgl2^{-/-} mice (23). As expected, fgl2^{Tg} macrophages showed equivalent prothrombinase activity to fgl2^{-/-} macrophages (FIG. 6). The expression of EGFP was observed in all organs examined, including the lymph nodes and the spleen of fgl2^{Tg} mice (FIG. 2C). By immunofluorescence, FGL2 was detected in all cells in fgl2^{Tg} mouse hearts but not in fgl2^{-/-} littermate hearts (FIG. 2D). By western blot, the expression of FGL2 was higher in fgl2^{Tg} mouse hearts than in fgl2^{+/+} mouse hearts (FIG. 2E). The western blot also confirmed that FGL2 and EGFP were both expressed in hearts from fgl2^{Tg} mice. On a single cell level, greater than 80% of T cells from fgl2^{Tg} mice expressed EGFP (FIG. 2F). in vitro, fgl2^{Tg} splenic mononuclear cells (SMNC) produced more FGL2 than fgl2^{+/+} or fgl2^{-/-} SMNC both unstimulated and following stimulation with IFNγ or ConA (FIG. 2G).

Characterization of Immune Function in fgl2^{Tg} Mice

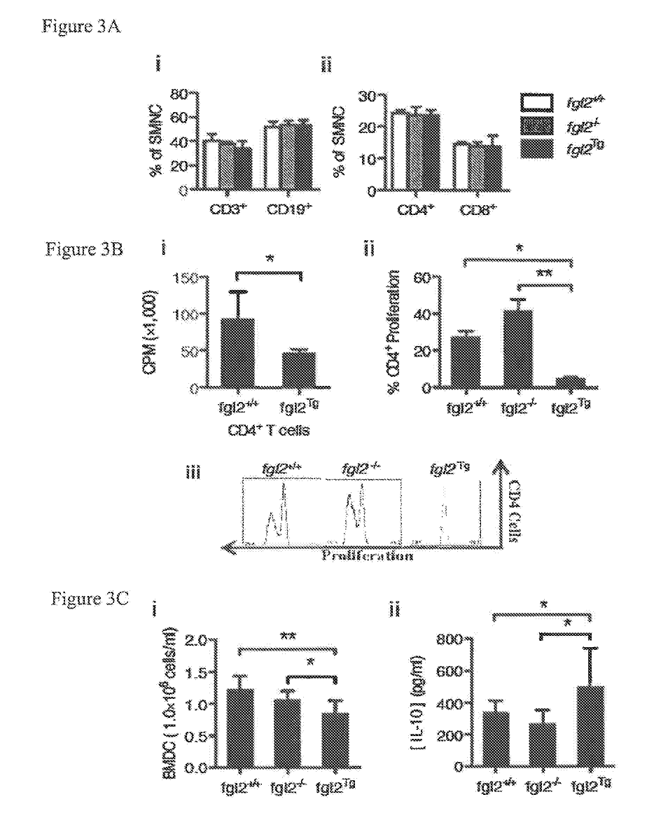

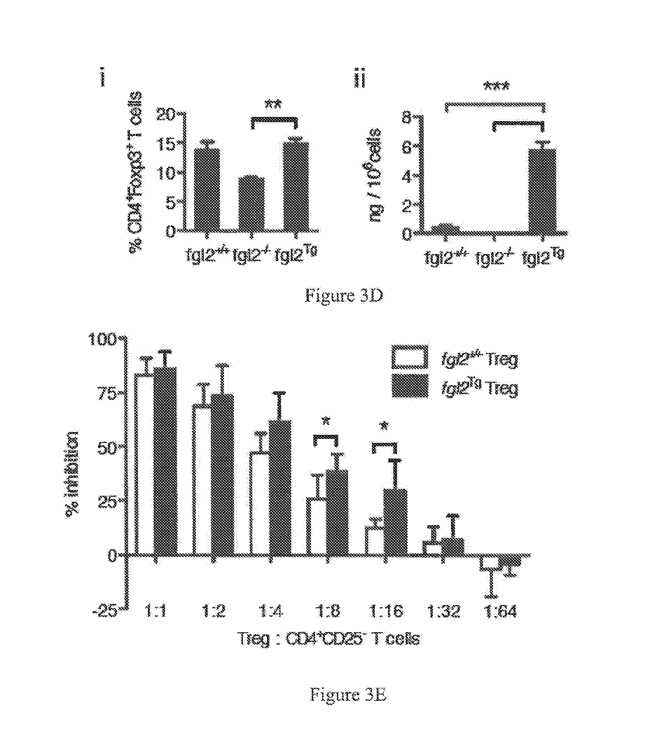

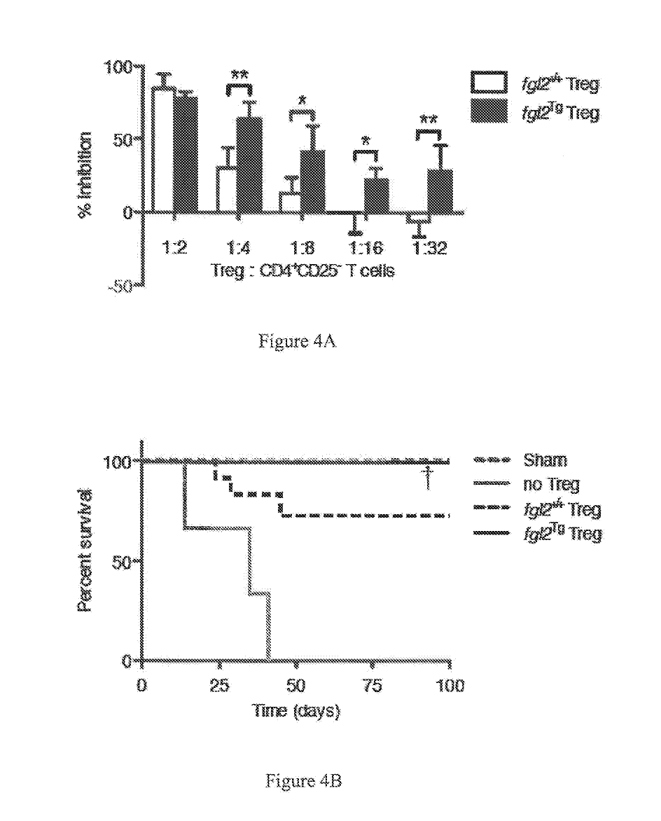

It was confirmed that absolute numbers of SMNC were similar among the fgl2^{Tg}, fgl2^{+/+}, and fgl2^{-/-} groups (data not shown), as were the subpopulations of CD19⁺ (B cells) and CD3⁺ (T cells) (FIG. 3Ai). Within the T cell compartment, the numbers of CD4⁺ and CD8⁺ T cells were also equivalent among the groups, indicating that there was no gross disturbance in lymphocyte development in fgl2^{Tg} mice (FIG. 3Aii). Given previous findings that fgl2^{-/-} T cells have increased proliferation to alloantigens (23), the activity of these cells in fgl2^{Tg} mice were explored. Fgl2^{Tg} CD4⁺ T cells had reduced proliferative responses to alloantigens and to anti-CD3 and anti-CD28 stimulation compared with fgl2^{+/+} or fgl2^{-/-} CD4⁺ T cells (FIG. 3Bi-iii). Previously, it has been shown that fgl2^{-/-} mice had increased numbers of DC and that fgl2^{-/-} BMDC had increased expression of MHCII and CD8((22, 23). The present disclosure demonstrates that the expansion of BMDC from fgl2^{Tg} was reduced compared with fgl2^{+/+} and fgl2^{-/-} BMDC (FIG. 3Ci). Furthermore, fgl2^{Tg} BMDC expressed higher levels of IL-10 than fgl2^{+/+} and fgl2^{-/-} BMDC (FIG. 3Cii). The numbers and function of fgl2^{Tg} Treg were then investigated. Splenic Treg as a percentage of CD4⁺ cells were increased in fgl2^{Tg} mice (FIG. 3Di): however, there was no difference in the absolute numbers of Treg in the spleens of fgl2^{+/+}, fgl2^{-/-} and fgl2^{Tg} mice (data not shown). To verify FGL2 overexpression by fgl2^{Tg} Treg, Treg with anti-CD3 and IL-2 were cultured. fgl2^{Tg} Treg produced significantly more FGL2 compared to fgl2^{+/+} or fgl2^{-/-} Treg (FIG. 3Dii). To evaluate the immunosuppressive activity of fgl2^{Tg} Treg, fgl2^{+/+} T cells were incubated with the T cell mitogen ConA, irradiated fgl2^{+/+} accessory cells and titrated numbers of fgl2^{Tg} or fgl2^{+/+} Treg. This assay demonstrated that fgl2^{Tg} Treg had increased immunosuppressive activity compared to fgl2^{+/+} Treg in response to ConA stimulation (FIG. 3E). fgl2^{Tg} Treg also had increased suppressive activity towards allogeneic responses at multiple Treg to T cells ratios in a MLR assay (FIG. 4A). To examine the in vivo function of fgl2^{Tg} Treg, fgl2^{+/+} CD4⁺CD25⁻ T cells were adoptively transferred with fgl2^{+/+} or fgl2^{Tg} CD4⁺CD25⁺ Treg into Rag1^{-/-} mice, The following day, Rag1^{-/-} mice received a fully mismatched BALB/c skin graft. Untreated Rag1^{-/-} mice accepted grafts long-term, and Rag1^{-/-} mice that only received fgl2^{+/+} T cells rejected skin grafts. Coadministration of either fgl2+/+ or fgl2^{Tg} Treg with fgl2^{+/+} T cells prevented rejection (no Treg vs. fgl2^{+/+} Treg, p=0,005; no Treg vs. fgl2^{Tg} Treg, p<0,001). There was a trend for fgl2^{Tg} Treg to have enhanced suppressive activity as there was 100% (11/11) graft survival with fgl2^{Tg} Treg and 75% (9/12) graft survival with fgl2^{+/+} Treg (p=0.07) (FIG. 4B).

### Fgl2^{Tg} Mice Develop Tolerance to Fully Mismatched Cardiac Grafts

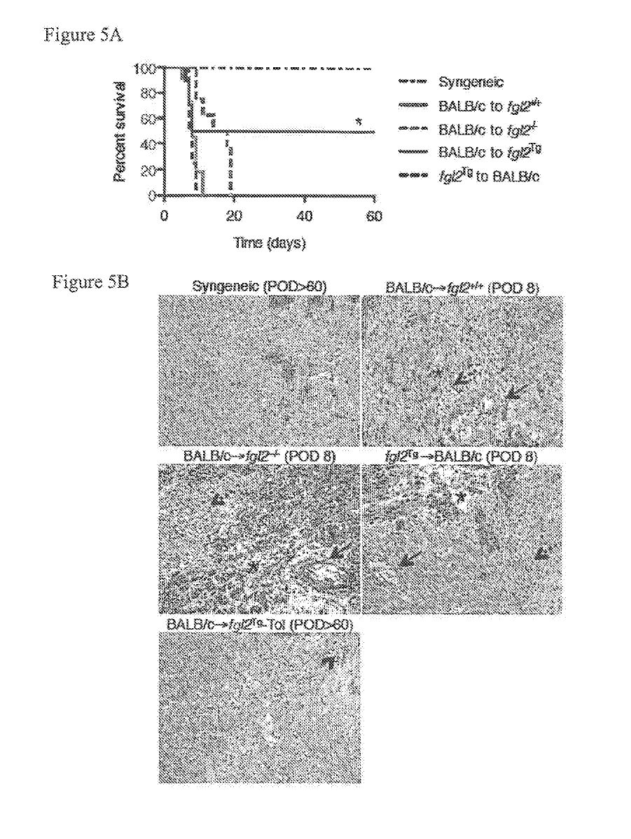

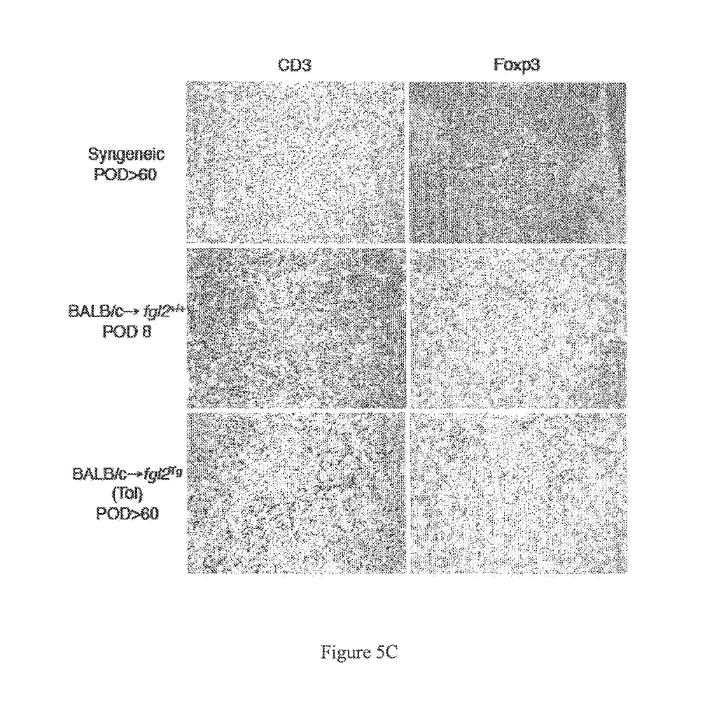

To determine if constitutive FGL2 overexpression could induce tolerance, BALB/cJ (H-2d) hearts were transplanted into fgl2^{Tg} (H-2^{b}) recipients. Half of the fgl2^{Tg} recipients rejected grafts with the same kinetics as fgl2^{+/+} mice. However, 50% of grafts transplanted into fgl2^{Tg} recipients survived indefinitely in the absence of immunosuppressive therapy (FIG. 5A). In comparison, BALB/c hearts transplanted into fgl2^{+/+} and fgl2^{-/-} recipients were rejected with a median rejection time of 8 and 7 days, respectively (FIG. 5A). Fgl2^{Tg} hearts were also transplanted into BALB/c recipients but were rejected at a median time of 8 days. Plasma levels of FGL2 were not significantly different between fgl2^{Tg} mice that rejected or accepted allografts (data not shown).

Tolerance is Associated with Increased Intragraft Treg and Reduced T Cell Responses

On histologic examination, syngeneic grafts and allografts in tolerant fgl2^{Tg} mice≦60 days post-transplant showed minimal cellular infiltrates with little evidence of cardiomyocyte necrosis or vasculitis (FIG. 5B), In contrast, allografts from rejecting fgl2^{Tg}, fgl2^{+/+}, and fgl2^{-/-} mice were fibrotic and contained areas of marked cellular infiltrates, severe vasculitis and cardiomyocyte necrosis (FIG. 5B). Immunostaining was performed to enumerate CD3⁺ T cells and Foxp3⁺ T cells in grafts (FIG. 5C). Allografts from tolerant fgl2^{Tg} mice had increased absolute numbers of Treg compared with allografts from rejecting fgl2^{Tg} and fgl2^{+/+} mice (FIG. 5D), Importantly, allografts from tolerant fgl2^{Tg} mice had significantly higher Foxp3⁺ to CD3⁺ T cell ratios compared with the other groups (FIG. 5Diii).

To examine the specificity of the tolerance, mixed lymphocyte reactions were performed in which responder SMNC were incubated with syngeneic (fgl2^{+/+}), donor-specific (BALB/c) or 3^{rd} party (CBA) stimulator cells. SMNC from non-transplanted fgl2^{Tg} and tolerant fgl2^{Tg} mice had reduced proliferation to both BALB/c and CBA stimulators compared with SMNC from non-transplanted fgl2^{+/+} mice. SMNC from tolerant fgl2^{Tg} mice had reduced proliferation to donor BALB/c stimulators but equivalent responses to CBA cells compared with SMNC from non-transplanted fgl2^{Tg} mice, demonstrating that tolerance was donor-specific (FIG. 5E).

### Discussion

The ability to induce tolerance would be a significant advance in the field of solid organ transplantation (7, 8). It may also assist in treating diseases or conditions wherein it is desirable to suppress an immune response, such as graft versus host disease, autoimmune diseases and allergies. Previously, it has been shown that FGL2-expressing Treg are increased in tolerant hearts in a mouse model of rapamycin-induced transplant tolerance and that treatment of tolerant mice with a monoclonal antibody to FGL2 led to rejection (11, 27, 28). The present disclosure describes the role of the Treg effector molecule FGL2 in the induction of transplantation tolerance,

The data disclosed herein demonstrates that recombinant FGL2 (rFGL2) treatment or constitutive FGL2 overexpression could promote transplant tolerance in mice. As shown herein, rFGL2 is able to prevent rejection of cardiac allografts, but following cessation of therapy, the cardiac allografts were rejected. The finding that allografts survived for up to 2 weeks following rFGL2 treatment is in agreement with the potent immunosuppressive activity of FGL2 (13, 19). To investigate if constitutive expression of FGL2 was able to promote tolerance, FGL2 overexpressing mice on a a fgl2^{-/-} background were generated. Fgl2^{Tg} mice had FGL2 plasma levels 7-fold higher compared to fgl2⁺¹⁺mice. All fgl2^{Tg} mice developed normally, had normal hematologic and biochemical lab values and showed no evidence of the glomerulonephritis seen in fgl2^{-/-} mice. Using a heterotopic heart transplant model, 50% of fgl2^{Tg} recipient mice were found to be tolerant to fully mismatched cardiac allografts. Tolerance was associated with increased Treg to CD3⁺ ratios in allografts and was donor-specific as SMNC from tolerant mice had reduced proliferation to donor but not third party antigens.

The data provided herein strongly supports the concept that FGL2-expressing Treg promotes tolerance. For one, constitutive secretion of FGL2 by the allograft was insufficient for tolerance induction as fgl2^{Tg} hearts transplanted into BALB/c recipients were universally rejected. In contrast, tolerance developed when recipient cells overexpressed FGL2 and was associated with increased numbers of intragraft Treg. In addition, fgl2^{Tg} Treg were more potent suppressors of alloimmune responses than fgl2^{+/+} Treg in vitro. It has previously been shown that FGL2-expressing Treg were present in tolerant allografts induced by rapamycin and nearly absent in rejecting allografts. Furthermore, induction of tolerance by rapamycin could be completely reversed by an anti-FGL2 antibody (28). Most recently, a report by Joller et al demonstrated that TIGIT⁺ Treg, a highly suppressive Treg subset, express large amounts of FGL2. In vivo, FGL2 was critical in the control of T cell expansion by TIGIT⁺ Treg in lymphopenic hosts and in controlling a number of inflammatory diseases including colitis (27).

It is unclear why only 50% of fgl2^{Tg} mice developed tolerance to allografts in the present study. Plasma levels of FGL2 in rejecting and tolerant mice were nearly identical and thus, variation in the plasma levels of FGL2 did not account for graft acceptance. However, the intragraft presence of Treg did appear to be necessary for maintaining tolerance as indicated by the near absence of Treg from fgl2^{Tg} mice that rejected grafts. The observation that tolerant fgl2^{Tg} mice had reduced MLR responses to donor but not third party antigens as compared with non-transplanted fgl2^{Tg} mice suggests that tolerant fgl2^{Tg} mice had an expansion of donor-specific Treg. Thus, the variation in the tolerance induction in fgl2^{Tg} mice may relate to factors that affect the expansion and homing of donor-specific Treg including the expression of chemokines and chemokine receptors (37, 38).

Additionally, tolerance in fgl2^{Tg} mice likely involved other cell types that could act synergistically with Treg. In prior studies, it has been demonstrated that fgl2⁻¹⁻ mice have evidence of abnormal immune activation of T cells, B cells and DC (19), Furthermore, the induction of apoptosis by FGL2 in DC and B cells was impaired in fgl2^{-/-} mice (19, 23). In the current study, it has been observed that fgl2^{Tg} CD4⁺ T cells were hypoproliferative in response to various stimuli. As the Treg to T cell ratio has been shown to be important in tolerance (37, 38), the reduced proliferation of fgl2^{Tg} T cells could have shifted the Treg to T cell ratio in favour of tolerance. Ongoing studies will distinguish whether impaired of fgl2^{Tg} T cell proliferation is due to an intrinsic T cell defect in mice constitutively overexpressing FGL2, or whether this is the result of an indirect feedback mechanism via ligation of the cognate receptor, FcγRIIB, expressed on antigen presenting cells (19). The studies disclosed herein also suggest that fgl2^{Tg} DC could have played a significant role in promoting tolerance. Fgl2^{Tg} BMDC displayed a tolerogenic phenotype with enhanced 1L-10 production compared with fgl.2′⁻'¹⁺BMDC. DC are known to play a critical role in tolerance induction, For instance, DC can shift immune responses to tolerance by inducing Treg (39-42). B regulatory cells may also play a role in tolerance as was recently shown in a rat transplant model (43). In that study, the authors used an adenovirus associated virus to overexpress FGL2 (AAV-FGL2) in recipients 30 days prior to transplantation and found that some of the recipients that received the AAV-FGL2 treatment developed tolerance. CD45RA+ cells from the tolerant recipients could transfer tolerance to sub-lethally irradiated recipients (43).

The present disclosure describes the generation of fgl2^{Tg} mice that express high levels of FGL2. It has been shown herein that 50% of fgl2^{Tg} mice accept fully mismatched heart allografts. Tolerance was associated with increased numbers and proportion of intragraft Treg. This disclosure demonstrates that FGL2 has potent immunosuppressive activity and supports the concept that expression of FGL2 by Treg promotes transplant tolerance, As such, approaches to specifically expand FGL2-expressing Treg may lead to transplant tolerance in humans.

All publications, patents and patent applications are herein incorporated by reference in their entirety to the same extent as if each individual publication, patent or patent application was specifically and individually indicated to be incorporated by reference in its entirety.

### Tables

Tables 1-2: Biochemical and Hematological characterization of fgl2^{Tg} mice. fgl2^{Tg} mice developed with similar biochemical and hematological parameters as littermate controls. For biochemical analysis, blood was collected by cardiac puncture from fgl2^{Tg} mice 6-10 weeks, 6-7 and 12 months of age. Blood samples were incubated at RT for 2 h and spun at 425×g. Serum was transferred to a fresh tube and stored at −80° C. Samples were sent to Charles River Laboratories for a biochemical analysis. Hematological analysis was performed on 50-60 μl of unclotted blood collected from the saphenous vein. Blood was collected into EDTA coated tubes and sent for analysis to the Toronto Centre for Phenogenomics.

TABLE 1
Summary of Hematological analysis for fgl2^{Tg} mice
2-3 months

| | | RBC | Hgb | HCT | MCV | MCH | MCHC | PLT | WBC | MPV | RDW | NE | LY | MO | BA | EO |
|---|---|---|---|---|---|---|---|---|---|---|---|---|---|---|---|---|
| | | (10¹²/L) | (g/L) | (L/L) | (fL) | (pg/cell) | (g/L) | (10°/L) | (10°/L) | (fL) | (%) | (10°/L) | (10°/L) | (10°/L) | (10°/L) | (10°/L) |
| fgl2^{-/-} | Aver- | 10.42 | 157.75 | 0.57 | 54.10 | 15.15 | 283.00 | 636.25 | 14.28 | 4.78 | 17.85 | 3.07 | 10.08 | 0.88 | 0.06 | 0.19 |
| | age | | | | | | | | | | | | | | | |
| | SD | 0.68 | 9.43 | 0.11 | 6.67 | 0.56 | 32.02 | 116.63 | 2.59 | 0.56 | 1.28 | 1.35 | 0.94 | 0.24 | 0.05 | 0.14 |
| fgl2^{Tg} | Aver- | 10.30 | 157.25 | 0.52 | 50.05 | 15.28 | 305.75 | 775.00 | 15.84 | 4.80 | 17.80 | 3.72 | 11.18 | 0.86 | 0.02 | 0.07 |
| | age | | | | | | | | | | | | | | | |
| | SD | 0.85 | 9.74 | 0.04 | 2.64 | 0.41 | 10.37 | 81.82 | 3.88 | 0.65 | 1.87 | 1.33 | 2.25 | 0.27 | 0.02 | 0.06 |
| | T-test | 0.84 | 0.94 | 0.40 | 0.30 | 0.73 | 0.23 | 0.10 | 0.53 | 0.96 | 0.97 | 0.52 | 0.40 | 0.93 | 0.14 | 0.15 |
| fgl2^{-/-} | Aver- | 10.84 | 157.33 | 0.51 | 47.17 | 14.53 | 308.33 | 777.00 | 14.72 | 4.50 | 18.53 | 3.46 | 9.99 | 0.98 | 0.08 | 0.21 |
| | age | | | | | | | | | | | | | | | |
| | SD | 0.99 | 13.05 | 0.06 | 2.45 | 0.25 | 13.80 | 31.22 | 3.57 | 0.35 | 1.05 | 0.45 | 2.45 | 0.38 | 0.11 | 0.26 |
| fgl2^{Tg} | Aver- | 10.69 | 152.67 | 0.51 | 47.97 | 14.30 | 298.33 | 796.33 | 14.33 | 4.83 | 17.50 | 3.05 | 9.83 | 1.00 | 0.13 | 0.33 |
| | age | | | | | | | | | | | | | | | |
| | SD | 0.94 | 13.65 | 0.06 | 1.50 | 0.10 | 9.24 | 90.51 | 2.93 | 0.06 | 0.44 | 1.00 | 2.08 | 0.41 | 0.07 | 0.09 |
| | T-test | 0.86 | 0.69 | 0.97 | 0.65 | 0.21 | 0.36 | 0.74 | 0.89 | 0.18 | 0.19 | 0.55 | 0.94 | 0.96 | 0.57 | 0.49 |
| fgl2^{-/-} | Aver- | 10.43 | 142.60 | 0.45 | 44.10 | 13.96 | 315.80 | 1008.00 | 14.17 | 4.64 | 18.30 | 3.32 | 10.14 | 0.58 | 0.03 | 0.10 |
| | age | | | | | | | | | | | | | | | |
| | SD | 0.83 | 18.01 | 0.05 | 0.93 | 0.77 | 15.61 | 90.39 | 2.00 | 0.28 | 0.45 | 1.38 | 1.30 | 0.12 | 0.05 | 0.11 |
| fgl2^{Tg} | Aver- | 10.65 | 145.60 | 0.46 | 44.44 | 14.18 | 319.20 | 1036.20 | 16.40 | 4.76 | 18.40 | 3.39 | 12.00 | 0.70 | 0.07 | 0.24 |
| | age | | | | | | | | | | | | | | | |
| | SD | 0.79 | 25.23 | 0.08 | 1.92 | 0.62 | 3.11 | 117.10 | 3.26 | 0.18 | 1.02 | 1.13 | 2.04 | 0.18 | 0.05 | 0.08 |
| | T-test | 0.68 | 0.83 | 0.90 | 0.73 | 0.63 | 0.65 | 0.68 | 0.23 | 0.44 | 0.85 | 0.93 | 0.12 | 0.25 | 0.35 | 0.04 |

Red Blood Cell (RBC), Hemoglobin (HGB), hematocrit (HCT), Mean Corpuscular (erythrocyte) volume (MCV), Mean Corpuscular (erythrocyte) hemoglobin, Mean Corpuscular (erythrocyte) hemoglobin concentration (MCHC), platelet/thrombocyte count (Plt), white blood cell/leukocyte count (WBC), Mean Platelet (thrombocyte) volume (MPV), Red cell (erythrocyte volume) Distribution Width (RDW), neutrophils (NE), lymphocytes (LY), monocytes (MO), basophils (BA), eosinophils (EO).

TABLE 2
Summary of biochemistry analysis for fgl2^{Tg} mice

| | Age | | | | | | | | | | | | | | | | |
|---|---|---|---|---|---|---|---|---|---|---|---|---|---|---|---|---|---|
| | | | 6-11 weeks | | | | | 6 months | | | | | 1 year | | | | |
| | C57BL/6* | | fgl2^{Tg} | | fgl2^{-/-} | | T- | fgl2^{Tg} | | fgl2^{-/-} | | T- | fgl2^{Tg} | | fgl2^{-/-} | | T- |
| Test | Low | High | Avg | SD | Avg | SD | test | Avg | SD | Avg | SD | test | Avg | SD | Avg | SD | test |
| Cholesterol | 69 | 169 | 113 | 19 | 126 | 34 | 0.55 | 111 | 14 | 100 | 26 | 0.56 | 122 | 14 | 127 | 33 | 0.79 |
| (mg/dl) | | | | | | | | | | | | | | | | | |
| Triglycerides | 67 | 278 | 75 | 14 | 98 | 18 | 0.10 | 110 | 33 | 76 | 5 | 0.15 | 97 | 48 | 88 | 40 | 0.76 |
| (mg/dl) | | | | | | | | | | | | | | | | | |
| Alanine | 28 | 129 | 36 | 7 | 37 | 3 | 0.71 | 88 | 86 | 53 | 19 | 0.53 | 43 | 7 | 40 | 8 | 0.46 |
| amino- | | | | | | | | | | | | | | | | | |
| transferase | | | | | | | | | | | | | | | | | |
| (u/l) | | | | | | | | | | | | | | | | | |
| Aspartate | 46 | 392 | 71 | 43 | 87 | 13 | 0.48 | 140 | 64 | 95 | 42 | 0.37 | 68 | 11 | 99 | 30 | 0.06 |
| amino- | | | | | | | | | | | | | | | | | |
| transferase | | | | | | | | | | | | | | | | | |
| (u/l) | | | | | | | | | | | | | | | | | |
| Alkaline | 111 | 275 | 101 | 38 | 121 | 35 | 0.46 | 56 | 8 | 72 | 14 | 0.15 | 57 | 8 | 51 | 11 | 0.32 |
| phosphatase | | | | | | | | | | | | | | | | | |
| (u/l) | | | | | | | | | | | | | | | | | |
| Bilirubin | 0.2 | 0.6 | 0.2 | 0.1 | 0.3 | 0.1 | 0.36 | 0.3 | 0.1 | 0.2 | 0.0 | 0.12 | 0.2 | 0.0 | 0.3 | 0.1 | 0.21 |
| (total) | | | | | | | | | | | | | | | | | |
| (mg/dl) | | | | | | | | | | | | | | | | | |
| Glucose | 172 | 372 | 197 | 25 | 197.3 | 41.1 | 0.99 | 173.3 | 55.0 | 163.3 | 63.1 | 0.85 | 204 | 35 | 178 | 52 | 0.39 |
| (mg/dl) | | | | | | | | | | | | | | | | | |
| Inorganic | 7.9 | 15 | 7.4 | 0.8 | 7.9 | 1.2 | 0.49 | 5.2 | 1.0 | 5.5 | 0.6 | 0.65 | 6.2 | 0.6 | 6.6 | 0.8 | 0.42 |
| phosphate | | | | | | | | | | | | | | | | | |
| (mg/dl) | | | | | | | | | | | | | | | | | |
| Total | 4.8 | 7.0 | 5.1 | 0.3 | 5.3 | 0.3 | 0.31 | 4.7 | 0.6 | 5.2 | 0.8 | 0.51 | 5.5 | 0.2 | 5.3 | 0.3 | 0.17 |
| Protein | | | | | | | | | | | | | | | | | |
| (g/dl) | | | | | | | | | | | | | | | | | |
| Calcium | 9.7 | 13 | 9.5 | 0.1 | 9.6 | 0.2 | 0.37 | 9.2 | 0.6 | 9.3 | 0.4 | 0.81 | 10.0 | 0.7 | 9.5 | 0.4 | 0.18 |
| (mg/dl) | | | | | | | | | | | | | | | | | |
| Blood Urea | 7 | 28 | 23 | 4 | 26 | 2 | 0.23 | 16 | 1 | 18 | 4 | 0.57 | 21 | 1 | 16 | 6 | 0.10 |
| Nitrogen | | | | | | | | | | | | | | | | | |
| (mg/dl) | | | | | | | | | | | | | | | | | |
| Creatinine | 0.2 | 0.5 | 0.3 | 0.1 | 0.3 | 0.1 | 0.62 | 0.2 | 0.1 | 0.2 | 0.1 | 0.42 | 0.3 | 0.0 | 0.3 | 0.1 | 0.92 |
| (mg/dl) | | | | | | | | | | | | | | | | | |
| Albumin | 2.8 | 3.8 | 2.9 | 0.2 | 3.1 | 0.1 | 0.17 | 2.9 | 0.4 | 2.8 | 0.6 | 0.88 | 3.0 | 0.1 | 2.9 | 0.2 | 0.25 |
| (g/dl) | | | | | | | | | | | | | | | | | |
| Sodium | 145 | 176 | 154.7 | 2.2 | 154.5 | 1.6 | 0.92 | 154.6 | 0.8 | 152.7 | 0.4 | 0.04 | 155.7 | 2.2 | 154.3 | 0.8 | 0.25 |
| (meq/l) | | | | | | | | | | | | | | | | | |
| Potassium | 7.6 | 11 | 4.26 | 0.58 | 4.65 | 0.45 | 0.33 | 6.05 | 0.45 | 5.28 | 0.48 | 0.17 | 4.51 | 0.67 | 5.54 | 0.56 | 0.04 |
| (meq/l) | | | | | | | | | | | | | | | | | |
| Chloride | 111 | 130 | 112 | 2 | 111.4 | 2.5 | 0.93 | 112.3 | 1.2 | 113.9 | 1.1 | 0.21 | 114 | 3 | 113 | 1 | 0.30 |
| (meq/l) | | | | | | | | | | | | | | | | | |

*Data derived from C57/BL6 Mouse Biochemistry as performed by Charles River

### REFERENCES

1. Sykes M, Levy G. Advances in transplantation. Seminars in immunology. 2011; 23(4):222-3.
2. Garcia G G, Harden P, Chapman J, World Kidney Day Steering C. The global role of kidney transplantation. Nephrology, dialysis, transplantation official publication of the European Dialysis and Transplant Association - European Renal Association. 2013; 28(8):5,
3. Gotthardt D N, Bruns H, Weiss K H, Schemmer P. Current strategies for immunosuppression following liver transplantation. Langenbeck's archives of surgery/Deutsche Gesellschaft fur Chirurgie. 2014.
4. Kaplan B, Qazi Y, Wellen J R. Strategies for the management of adverse events associated with mTOR inhibitors. Transplantation reviews (Orlando, Fla). 2014.
5. Heemann U, Lutz J. Pathophysiology and treatment options of chronic renal allograft damage. Nephrology, dialysis, transplantation : official publication of the European Dialysis and Transplant Association - European Renal Association. 2013; 28(10):2438-46,
6. Savasta M, Lentini S. Immunology insights into cardiac allograft rejection. Reviews in cardiovascular medicine. 2011;12(2):76.
7. Sachs D, Sykes M, Kawai A, Cosimi. Immuno-intervention for the induction of transplantation tolerance through mixed chimerism. Seminars in immunology. 2011; 23(3): 165-73.
8. McMurchy A N, Bushell A, Levings M K, Wood K J. Moving to tolerance: clinical application of T regulatory cells. Seminars in immunology. 2011;23(4):304-13.
9. Issa F, Wood K J. CD4+ regulatory T cells in solid organ transplantation. Current opinion in organ transplantation. 2010; 15(6):757-64.
10. Sakaguchi S. The origin of FOXP3-expressing CD4+regulatory T cells: thymus or periphery. The Journal of clinical investigation. 2003; 112(9):1310-2.
11. Li X, Menoret S, Bezie S, Caron L, Chabannes D, Hill M, et al. Mechanism and localization of CD8 regulatory T cells in a heart transplant model of tolerance. Journal of immunology (Baltimore, Md: 1950). 2010; 185(2):823-33.
12. Levings M, Gregori S, Tresoldi E, Cazzaniga S, Bonini C, Roncarolo M. Differentiation of Trl cells by immature dendritic cells requires 1L-10 but not CD25+CD4+ Tr cells. Blood. 2005; 105(3):1162-9.
13. Shevach E. Mechanisms of foxp3+ T regulatory cell-mediated suppression. Immunity. 2009; 30(5):636-45.
14. Wood K, Sakaguchi S. Regulatory T cells in transplantation tolerance. Nature reviews Immunology. 2003; 3(3):199-210.
15. Goto R, You S, Zaitsu M, Chatenoud L, Wood KJ. Delayed anti-CD3 therapy results in depletion of alloreactive T cells and the dominance of Foxp3+ CD4+ graft infiltrating cells. American journal of transplantation: official journal of the American Society of Transplantation and the American Society of Transplant Surgeons. 2013; 13(7)1 655-64.
16. Tang Q, Bluestone J A, Kang S-M M. CD4(+)Foxp3(+) regulatory T cell therapy in transplantation. Journal of molecular cell biology. 2012; 4(1):11-21.
17. Waldmann H, Hilbrands R, Howie D, Cobbold S. Harnessing FOXP3+ regulatory T cells for transplantation tolerance. The Journal of clinical investigation. 2014; 124(4)1439-45.
18. Fontenot J, Rasmussen J, Gavin M, Rudensky A. A function for interleukin 2 in Foxp3-expressing regulatory T cells. Nature immunology. 2005; 6(11):1142-51.
19. Liu H, Shalev I, Manuel J, He W, Leung E, Crookshank J, et al. The FGL2-FcgammaRIIB pathway: a novel mechanism leading to immunosuppression. European journal of immunology. 2008; 38(11):3114-26.
20. Marsden P, Ning O, Fung L, Luo X, Chen Y, Mendicino M, et al. The Fgl2/fibroleukin prothrombinase contributes to immunologically mediated thrombosis in experimental and human viral hepatitis. The Journal of clinical investigation. 2003; 112(1):58-66.
21. Yuwaraj S, Ding J, Liu M, Marsden P, Levy G. Genomic characterization, localization, and functional expression of FGL2, the human gene encoding fibroleukin: a novel human procoagulant. Genomics. 2001; 71(3):330-8.
22. Chan C, Kay L, Khadaroo R, Chan M, Lakatoo S, Young K, et al. Soluble fibrinogen-like protein 2/fibroleukin exhibits immunosuppressive properties: suppressing T cell proliferation and inhibiting maturation of bone marrow-derived dendritic cells. Journal of immunology (Baltimore, Md. 1950). 2003; 170(8):4036-44.
23. Shalev I, Liu H, Koscik C, Bartczak A, Javadi M, Wong K, et al. Targeted deletion of fgI2 leads to impaired regulatory T cell activity and development of autoimmune glomerulonephritis. Journal of immunology (Baltimore, Md: 1950). 2008; 180(1):249-60.
24. Herman A, Freeman G, Mathis D, Benoist C. CD4+CD25+ T regulatory cells dependent on ICOS promote regulation of effector cells in the prediabetic lesion. The Journal of experimental medicine. 2004; 199(11):1479-89.
25. Shalev I, Wong K, Foerster K, Zhu Y, Chan C, Maknojia A, et al. The novel CD4+CD25+ regulatory T cell effector molecule fibrinogen-like protein 2 contributes to the outcome of murine fulminant viral hepatitis. Hepatology (Baltimore, Md.).

2009; 49(2):387-97,
26. Denning T, Granger S, Mucida D, Graddy R, Leclercq G, Zhang W, et al. Mouse TCRalphabeta+CD8alphaalpha intraepithelial lymphocytes express genes that down-regulate their antigen reactivity and suppress immune responses. Journal of immunology (Baltimore, Md.: 1950). 2007; 178(7):4230-9,
27. Joller N, Lozano E, Burkett PR, Patel B, Xiao S, Zhu C, et al. Treg cells expressing the coinhibitory molecule TIGIT selectively inhibit proinflammatory Th1 and Th17 cell responses. Immunity. 2014; 40(4):569-81.
28. Urbanellis P, Shyu W, Khattar R, Wang J, Zakharova A, He W, et al, The Treg effector molecule fibrinogen-like protein 2 is necessary for the development of rapamycin-induced tolerance to fully MHC-mismatched murine cardiac allografts. Immunology. 2014.
29. Corry R, Winn H, Russell P. Primarily vascularized allografts of hearts in mice. The role of H-2D, H-2K, and non-H-2 antigens in rejection. Transplantation. 1973; 16(4):343-50,
30. Novak A, Guo C, Yang W, Nagy A, Lobe C. Z/EG, a double reporter mouse line that expresses enhanced green fluorescent protein upon Cre-mediated excision. Genesis (New York, N.Y. ; 2000). 2000; 28(3-4):147-55,
31. Lobe C, Koop K, Kreppner W, Lomeli H, Gertsenstein M, Nagy A. ZiAP, a double reporter for cre-mediated recombination. Developmental biology. 1999; 208(2):281-92.
32. Ma X-ZZ, Bartczak A, Zhang J, Khattar R, Chen L, Liu M F, et al. Proteasome inhibition in vivo promotes survival in a lethal murine model of severe acute respiratory syndrome. Journal of virology. 2010; 84(23):12419-28.
33. Inaba K, Swiggard W J, Steinman R M, Romani N, Schuler G, Brinster C. Isolation of dendritic cells. Curr Protoc Immunol, 2009 August; Chapter 3:Unit 37.
34. De Winter H, Cheroutre H, Kronenberg M. Mucosal immunity and inflammation. II. The yin and yang of T cells in intestinal inflammation: pathogenic and protective roles in a mouse colitis model. The American journal of physiology. 1999; 276(6 Pt 1):21.
35. Long E T, Baker S, Oliveira V, Sawitzki B, Wood K J. Alpha-1 ,2-mannosidase and hence Nglycosylation are required for regulatory T cell migration and allograft tolerance in mice. PloS one. 2010;5(1).
36. Lakso M, Pichel J, Gorman J, Sauer B, Okamoto Y, Lee E, et al. Efficient in vivo manipulation of mouse genomic sequences at the zygote stage. Proceedings of the National Academy of Sciences of the United States of America. 1996; 93(12):5860-5.
37. Zhang N, Schroppel B, Lal G, Jakubzick C, Mao X, Chen D, et al. Regulatory T cells sequentially migrate from inflamed tissues to draining lymph nodes to suppress the alloimmune response. Immunity. 2009; 30(3):458-69.
38. Fan Z, Spencer J A, Lu Y, Pitsillides CM, Singh G, Kim P, et al. In vivo tracking of ‘color-coded’ effector, natural and induced regulatory T cells in the allograft response. Nature medicine. 2010; 16(6):718-22.
39. Manicassamy S, Pulendran B. Dendritic cell control of tolerogenic responses. Immunological reviews. 2011.
40. Raich-Regue D, Glancy M, Thomson A W. Regulatory dendritic cell therapy: from rodents to clinical application. Immunology letters. 2014; 161(2):216-21.
41. Wood K J, Bushell A, Hester J. Regulatory immune cells in transplantation. Nature reviews Immunology. 2012;12(6):417-30.
42. Ezzelarab M, Thomson A W. Tolerogenic dendritic cells and their role in transplantation. Seminars in immunology. 2011;23(4):252-63.
43. Bezie S, Picarda E, Tesson L, Renaudin K, Durand J, Menoret S, et al. Fibrinogen-Like Protein 2/Fibroleukin Induces Long-Term Allograft Survival in a Rat Model through Regulatory B Cells. PloS one. 2015;10(3).
44. Duncan A R, Winter G. The binding site for C1q on IgG. Nature. 1988; 332(6166):738-40.
45. Clark D, Foerster K, Fung L, He W, Lee L, Mendicino M, et al. The fgl2 prothrombinaseifibroleukin gene is required for lipopolysaccharide-triggered abortions and for normal mouse reproduction. Molecular human reproduction. 2004; 10(2):99-108.
46. Ghanekar A, Mendicino M, Liu H, He W, Liu M, Zhong R, et al. Endothelial induction of fgl2 contributes to thrombosis during acute vascular xenograft rejection. Journal of immunology (Baltimore, Md.: 1950). 2004; 172(9):5693-701.
47. Hanington P C, Zhang S-M M. The primary role of fibrinogen-related proteins in invertebrates is defense, not coagulation. Journal of innate immunity. 2011; 3(1):17-27.
48. Doolittle RF. THE STRUCTURE AND EVOLUTION OF VERTEBRATE FIBRINOGEN. Annals of the New York Academy of Sciences. 1983;40
49. Marazzi, S., S. Blum, R. Hartmann, D. Gundersen, M. Schreyer, S. Argraves, V. von Fliedner, R. Pytela, and C. Ruegg. 1998. Characterization of human fibroleukin, a fibrinogen-like protein secreted by T lymphocytes. J Immunol 161:138-147.

1. A regulatory T cell (Treg) comprising a recombinant fgl2 transgene,

2. The Treg of claim 1, wherein the transgene is operably connected to a promoter that drives high levels of gene expression.

3. The Treg of claim 1, wherein the fgl2 transgene encodes a FGL2 protein that lacks prothrombinase activity.

4. The Treg of claim 3, wherein the fgl2 transgene comprises a nucleic acid sequence with at least 80%, 85%, 90%, 95%, 96%, 97%, 98%, 99% or 100% sequence identity to SEQ D NO:1, and further wherein the promoter is a CAG promoter.

5. A transgenic mouse whose genome comprises a recombinant fgl2 transgene, wherein the mouse ubiquitously expresses FGL2 protein.

6. The mouse of claim 5, wherein the transgene is operably connected to a promoter that drives high levels of gene expression.

7. The mouse of claim 5, wherein the fgl2 transgene encodes a FGL2 protein that lacks prothrombinase activity.

8. The mouse of claim 5, wherein the fgl2 transgene comprises the nucleic acid sequence with at least 80%, 85%, 90%, 95%, 96%, 97%, 98%, 99% or 100% sequence identity to SEQ ID NO:1, and further wherein the promoter is a CAG promoter.

9. The mouse of claim 5, wherein the mouse is humanized.

10. A Treg isolated from the mouse of claim 5.

11. A composition comprising the Treg of claim 1 and a pharmaceutically acceptable carrier.

12. The composition of claim 11, further comprising one or more immunosuppressive agents.

13. A method of inducing tolerance in a subject in need thereof comprising administering to the subject a therapeutically effective amount of the Treg of claim 1.

14. The method of claim 13 to induce tolerance to a transplanted organ or tissue.

15. The method of claim 14, wherein the Treg or composition is administered prior to, during or after the transplantation of the organ or tissue.

16. The method of claim 13 to induce tolerance for the treatment of graft versus host disease.

17. The method of claim 13 to induce tolerance for the treatment of an autoimmune disease or disorder.

18. The method of claim 13 to induce tolerance for the treatment of an allergic reaction.

19. The method of claim 13, wherein the subject is a mammal.

20. The method of claim 13, wherein the subject is a human.
